(19) United States
(12) Patent Application Publication
(10) Pub. No.: US 2015/0191714 A1
(43) Pub. Date: Jul. 9, 2015

(54) FORMULATIONS OF RECOMBINANT HUMAN BILE SALT-STIMULATED LIPASE

(71) Applicant: Swedish Orphan Biovitrum AB(Publ), Stockholm (SE)

(72) Inventor: Vilhelm Ek, Stockholm (SE)

(73) Assignee: Swedish Orphan Biovitrum AB(Publ), Stockholm (SE)

(21) Appl. No.: 14/346,296

(22) PCT Filed: Sep. 25, 2012
(86) PCT No.: PCT/SE2012/051011

§ 371 Date: Mar. 20, 2014

(30) Foreign Application Priority Data
Sep. 26, 2011 (SE) 1150874-4

(51) Int. Cl. C12N 9/96; C12N 9/20

(52) U.S. Cl. CPC C12N 9/96; C12N 9/20; C12Y 301/01013

(57) ABSTRACT

The present invention relates to improved formulations of recombinant human bile salt stimulated lipase (rhBSSL), including those suitable for forming a lyophilized formulation of rhBSSL, lyophilized formulations of rhBSSL per-se, unit dose forms of rhBSSL and reconstituted formulations of rhBSSL. The formulations of the present invention comprises rhBSSL, a crystalline bulking agent and an amorphous stabilizer that is a different chemical entity to said crystalline bulking agent. The formulations of the present invention have one or more desired properties, including those that relate to stability, decreased aggregation and/or formation of insoluble aggregates in solution. The lyophilized formulations of the present invention have pharmaceutical utility, particularly for the administration of rhBSSL to human infants.

### TECHNICAL FIELD

The present invention relates to improved formulations of recombinant human bile salt-stimulated lipase (rhBSSL), including those suitable for forming a lyophilized formulation of rhBSSL, lyophilized formulations of rhBSSL per-se, unit dose forms of rhBSSL and reconstituted formulations of rhBSSL. The formulations of the present invention have one or more desired properties, including those that relate to stability, decreased aggregation and/or formation of insoluble aggregates in solution. The lyophilized formulations of the present invention have pharmaceutical utility, particularly for the administration of rhBSSL to human infants.

### BACKGROUND ART

In adults, colipase-dependent pancreatic lipase (PTL) is the main enzyme responsible for the digestion of dietary triglycerides (TG). In the newborn infant, and particularly in the preterm infant, exocrine pancreatic functions are not fully developed (Manson & Weaver, 1997; Arch Dis Child Fetal Neonatal Ed, 76: 206-211). Hence, in the infant, expression of pancreatic lipases is low compared to adult pancreas (Lombardo, 2001; Biochim Biophys Acta, 1533: 1-28; Li et al 1007; Pediatr Res, 62: 537-541), the intraluminal PTL activity during established fat digestion is much lower compared to adults (Fredrikzon et al, 1978; Paediatr Res, 12: 138-140) and fat malabsorption is not uncommon (Carnielli et al, 1998; Am J Clin Nutr 67: 97-103; Chappell et al, 1986; J Pediatr, 108: 439-443). Lindquist and Hernell (1990; Curr Opin Clin Nutr Metab Care, 13: 314-320) have reviewed the subject of lipid digestion and absorption in early life.

At birth the human fetus switches from a glucose-dominated to a lipid-dominated energy supply since fat, or more specifically TG, that constitutes half of the total energy in human milk and most infant formulae, serves as the dominating energy substrate for newborn infants Therefore, efficient digestion and absorption of dietary TG is crucial to infant growth and development. In the breastfed infant, low PTL activity is compensated for by a broad-specificity lipase, bile salt-stimulated lipase (BSSL) (EC 3.1.1.13), which is secreted both from the lactating mammary gland into the milk and from the exocrine pancreas. In preterm infants, breast milk seems to provide the major part of BSSL in duodenal content during a breast milk meal (Fredrikzon et al, 1978), and breast-fed infants digest and absorb fat, and importantly long long-chain polyunsaturated fatty acids (LCPUFAs), more efficiently than formula-fed infants (Bernbäck et al, 1990; J Clin Invest, 85:1221-1226; Carnielli et al, 1998).

The superiority of human milk as a nutritional source for term as well as preterm infants has been manifested in many studies and expert group recommendations. Accordingly, the recommended feeding method world-wide is breastfeeding. Neither is however, breastfeeding nor feeding the mother's own breast milk always possible or recommended for medical reasons, and breastfeeding may not be practiced for a number of other reasons. In cases where the infant is not breast-fed, infant formula or banked and non-banked pasteurized and/or frozen breast milk is often used. All are, however, in some respects nutritionally suboptimal for newborn infants.

Due to risks of viral infection (human immunodeficiency virus [HIV], cytomegalovirus [CMV], hepatitis) and to a lesser degree transmission of pathogenic bacteria, donor milk used in so-called milk banks is generally pasteurized before it is used. However, BSSL is inactivated during pasteurization of human milk (Björksten et al, 1980; Br Med J, 201: 267-272); nor is it present in any of the many different formulas that exist for the nutrition of pre- or full-term neonates. It has been shown that fat absorption, weight gain and linear growth is higher in infants fed fresh compared to pasteurized breast milk (Andersson et al. 2007; Williams et al, 1978; Arch Dis Child 43: 555-563). This is one reason why it has been advocated that newborn infants, particularly preterm infants, that cannot be fed their own mother's milk should be fed non-pasteurized milk from other mothers (Björksten et al, 1980).

Native human milk BSSL (hBSSL-MAM) has been purified to homogeneity, as reported by Bläckberg and Hernell (1981; Eur J Biochem, 116: 221-225) and Wang & Johnson (1983), and the cDNA sequence of human BSSL was identified by Nilsson (1990; Eur J Biochem, 192: 543-550) and disclosed in WO 91/15234 and WO 91/18923. Characterization and sequence studies from several laboratories concluded that the proteins hBSSL-MAM and the pancreas carboxylic ester hydrolase (CEH) (also known as pancreatic BSSL) are both products of the same gene (for example, Baba et al, 1991; Biochem, 30: 500-510 Hui et al, 1990; FEBS Lett, 276: 131-134; Reue et al, 1991; J Lipid Res, 32: 267-276). Following the isolation of the cDNA sequence, rhBSSL, as well as variants thereof, has been produced including in transgenic sheep (rhBSSL-OVI); such as described in U.S. Pat. No. 5,716,817, WO 94/20610 and WO 99/54443.

Andersson et al, 2007 (Acta Paediatr 96: 1445-1449) reported a randomized study that shown pasteurization of mother's own milk reduced fat absorption and growth in preterm infants, and proposed that these effects were due to inactivation of milk-based BSSL by pasteurization. For recently, two randomized and controlled clinical trial have reported that addition of rhBSSL to pasteurized breast milk or to infant formula and administration to preterm infants showed statistically significant improvement in growth velocity of such infants. For example, as presented by Maggio et al at “The Power or Programming Conference 2010: International conference on developmental origins of health and disease”, Munich, May 6-8, 2010, and as presented by Carnielli et al at “The 3^{rd} congress of the European Society of Paediatric Societies”, Copenhagen, Oct. 24, 2010). The latter presentation also reported a small but not statistically significant increase in total coefficient of fat absorption (CFA) and a trend towards improved intestinal absorption of docosahexanoic acid (DHA) and arachidonic acid (AA)—two medically and developmentally important long chain polyunsaturated fatty acids (LCPUFAs)—when rhBSSL was added to infant formula compared to infant formula with placebo.

As described above, BSSL from breast milk is the same lipase as CEH produced by the mature human pancreas which is important for TG digestion in adults. Accordingly, rhBSSL has also been explored as a therapy for exocrine pancreatic insufficiency (PI) due to chronic pancreatitis or cystic fibrosis (CF) in human adults (e.g., Strandvik et al, 2004; 18th North American Cystic Fibrosis Conference, St. Louis, Mich.; abstract published in Pediatr Pulmonol, S27: 333). More recently, it has been announced that a further phase II trial with an oral suspension of rhBSSL (described therein as “bucelipase alpha”), dosed at 170 mg 3 times daily for 5-6 days, to evaluate the effect on the fat absorption in adult patients with CF and PI has been completed (clinicaltrials.gov identifier NCT00743483), and results presented at the 34^{th} European Cystic Fibrosis Society Conference, Hamburg, Germany, 8 to 11 Jun. 2011.

With growing evidence of therapeutic utility for rhBSSL, it would be desirable to provide formulations of rhBBSL for pharmaceutical uses that have particular properties, and/or are useful for administration to adults or to infants, in particular to preterm infants. For example, it would be desirable to provide formulations of rhBBSL that show improved or prolonged stability, such as in terms of reduced formation of high molecular weight aggregates; have practical or convenient storage conditions; have more reliable and uniform reconstitution properties; show a longer shelf life; have characteristics suitable for manufacturing or packaging; and/or possess other desirable properties.

Therapeutic proteins are commonly provided as a lyophilized formulation, comprising an amount of the protein of interest together with one or more pharmaceutical excipients such as a bulking agent, stabilizing agent, and/or salts. Lyophilization (also called freeze-drying) refers to a process that uses low temperature and pressure to remove a solvent, typically water, from a liquid formulation by the process of sublimation (i.e., a change in phase from solid to vapor without passing through a liquid phase). Lyophilization typically comprises three general steps: (1) freezing; (2) primary drying; and (2) secondary drying.

Freeze-drying is generally thought to be disruptive to the biological activity of biomolecules such as proteins. The magnitude of damage varies considerably with different biomolecules and different conditions, and various investigators have studied different systems. The freezing of aqueous solutions can create an initial increase in solute concentrations or pH that can be more damaging to labile proteins than the freezing itself. Bulking agents can be used to seek to enhance the formation and drying capability of the solid cake, or to improve its pharmaceutical elegance. Stabilizing agents can be used to seek to stabilize the activity of the biomolecule, but have limited and varying degrees of success, depending on the system. Crowe, et al (1987; Biochem J; 242: 1-10) describes the stabilization of dry phospholipid bilayers and proteins by sugars, and also reviews the understanding of the mechanisms of trehalose stabilization of cells in “The trehalose myth revisited: Introduction to a symposium on stabilization of cells in the dry state” Cryobiology 43, 89-105 (2001).

Various researchers have reported on using various excipients and combinations of excipients to protect various biomolecules, including the following examples.

WO 03/009817 describes the use of mannitol or glycine as bulking agents to form stable lyophilized formulations of IGG antibodies.

WO 2006/075072 describes freeze-dried formulations of various enzymes, including of a lipase, for use in a biosensor. Glycine and mannitol are used therein as crystalline bulking agents, while the protein is generally in an amorphous state.

In EP 1 932 519, describes lyophilized formulations of bone morphogenic proteins, particularly of recombinant human Growth and Differentiation Factor (rhGDF), including those comprising mannitol , and a separate formulation of rhGDF comprising glycine at about pH 4.

WO 2006/023665 describes IL-1 antagonist formulations, including a pre-lyophilization formulation comprising 5-50 mg/mL or protein and 0.25-3.0% of glycine as a lyoprotectant.

Chang et al (1996; Pharm Res, 13: 243-249) describe the development of a stable freeze-dried formulation of recombinant human Interlukin-1 Receptor Antagonist (rhIL-1ra), including testing mannitol or glycine as a bulking agent used in combination with an amorphous protein stabilizer, especially sucrose.

Hirakura et al (2004; Int J Pharm; 386: 53-67 investigated the impacts of temperature changes during the freezing processes on a lyophilized formulation containing sodium phosphate (10 mM, pH 7.0) and glycine (300 mM) of recombinant human Interleukin-11 (rhIL-11; 5 mg/mL).

Leuckel et al (1998; Pharm Dev and Tech 3: 325-336) investigated glycine, lysine-HCl or mannitol as crystallizing bulking agents in combination with the amorphous stabilizing agents sucrose or trehalose on the properties of the freeze-concentrate and the lyophisate. In the absence of any protein, depending on the particular combination of excipients and their concentration ratio, one or other of the excipients was able to form crystals.

Meyer et al (2009; Eur J Pharm Sci, 38: 29-38) studied the impact of bulking agents on the stability of a lyophilized anti-TNF murine monoclonal IgG. Combinations of sucrose as stabilizing agent with the bulking agents mannitol or glycine were evaluated for their effects on antibody stability.

Tian et al (2007; Int J Pharmac, 335: 20-31) evaluated the stabilization of humanized monoclonal antibodies in amino acid formulations. The protective effects of histidine, arginine, glycine or aspartic acid in anti CD11a and anti-IgE antibodies were tested.

WO 99/27983 describes a one-dose syringe containing a freeze-dried formulation of human growth hormone that less “blow-out” when used. Each unit-dose contained less than 1.4 mg protein, and various ratios of other excipients including about 0.2 mg glycine and 1.1 mg mannitol per mg of protein, and further including sodium- and disodium-phosphate.

WO 2006/081320 describes a liquid formulation suitable for freeze-drying that comprises at least 20 mg/mL protein, a crystalline bulking agent and amorphous solute at a weight:weight ratio of less than 1 Mannitol and glycine are described therein as being conventional “crystalline bulking agents”, but also that they may be used as a stabilizing agent, providing they remain in amorphous state following the freeze-drying process. One example included at least 2.0% w/v of the stabilizing agent in the liquid formulation.

US 2006/0275306 describes various lyophilized anti-IgG or anti-HER2 antibody formulations obtained from liquid formulations, including those comprising 21 mg/mL antibody with mannitol/glycine at 250/25 mM or 55/276 mM, respectively.

Pyne et al (2003; J Pharm Sci 92: 2172-2283) studied solute crystallization in mannitol-glycine systems and its implications on protein stabilization in freeze-dried formulations. The formation of mannitol and/or glycine crystals in the frozen material was affected by various factors including the rate of freezing, the relative concentrations of the mannitol and glycine in the liquid pre-lyophilized formulation and the presence/concentration of phosphate buffer.

WO 2007/112757 discloses processes for concentration of polypeptides including recombinant human porphobilinogen deaminase (rhPBGD) to form lyophilized formulations of such protein from various liquid formulations including a bulk solution comprising 3.67 mM Na₂HPO₄, 27 mM glycine, 250 mM mannitol at pH 7.9 (pH range 7.5 to 8.5).

These investigators report varying degrees of success using various different excipients or combinations thereof, as measured by various methods on various biomolecules and proteins. None of these investigators have reported on formulations of rhBSSL.

EP 0 317 355 generically discloses a dietary composition comprising a nutritional base from a first source, the base containing fats and being poor in bile salt-stimulated lipase; and an effective amount of bile salt-stimulated lipase from a second source.

WO 91/18923 generically discloses a pharmaceutical composition comprising recombinant human bile salt-stimulated lipase, WO 94/20610 generically claims a pharmaceutical composition comprising variants of human bile salt-stimulated lipase, and WO 99/54443 generically discloses a pharmaceutical composition comprising human bile salt-stimulated lipase produced from a transgenic animal. In each case, it is described that such pharmaceutical compositions may be used for the improvement of the utilization of dietary lipids in preterm born infants or for the treatment of a pathological conditions related to pancreatic insufficiency, e.g. in cystic fibrosis.

Co-pending WO 2012/052059 and WO 2012/052060 (the contents of which are hereby incorporated by reference in their entirety) disclose the preparation and use of a pharmaceutical composition of recombinant human bile salt-stimulated lipase to increase the growth rate and/or increase absorption of certain LCPUFDAs in pre-term human infants. The unit dose disclosed therein was a frozen oral solution comprising 15 mg/mL recombinant human bile salt-stimulated lipase dissolved in 1.3 mL water for injection. The unit dose was prepared from aliquots of a solution made from lyophilized bulk recombinant human bile salt-stimulated lipase dissolved in water for injection. Briefly, the lyophilized bulk recombinant human bile salt-stimulated lipase was obtained by production of the protein using recombinant CHO cells, purification of the recombinant protein from the cells using a variety of steps including anion exchange chromatography, diafiltration, concentration, and finally freeze-drying. The lyophilized formulation and finished unit dose further comprised sodium dihydrogen phosphate and sodium chloride as rhBSSL drug substance was lyophilized from a phosphate/sodium chloride buffered bulk solution of rhBSSL.

Thus, there is conflicting evidence on what is an optimal combination of excipients to afford lyoprotection of biomolecules such as proteins, and no specific guidance as to those to use in or to form lyophilized formulations comprising rhBSSL. There is not any one combination of excipients that is optimal for all proteins, but rather a significant degree of experimentation is required to obtain the desired results for the protein under investigation. There remains a need for one or more, or a combination of, pharmaceutically acceptable excipients suitable for rhBSSL, including those that protect the protein during lyophilization, storage, and/or use, or that provide other desirable properties including shelf-life, manufacturing and/or reconstitution characteristics, or one or more other property as described herein.

### BRIEF DESCRIPTION OF THE INVENTION

The solution to one or more of the above technical problems is provided by the various aspects and embodiments of the present invention as defined or otherwise disclosed herein and/or in the claims. Generally, and by way of brief description, the main aspects of the present invention can be described as follows:

In one aspect, the invention relates to a formulation suitable for lyophilization comprising recombinant human bile salt-stimulated lipase (rhBSSL); a crystalline bulking agent; and an amorphous stabilizer that is a different chemical entity to said crystalline bulking agent.

In another aspect, the invention relates to a lyophilized formulation obtainable by lyophilization of a liquid formulation of the present invention, where said lyophilized formulation comprises rhBSSL, a crystalline bulking agent and an amorphous stabilizer. In a related aspect, the invention also relates to a unit dose of such a lyophilized formulation. In other related aspects, the present invention further relates to a method of producing such a lyophilized formulation, and also to a lyophilized formulation obtainable by such method.

In yet another aspect, the invention relates to a method of producing a reconstituted formulation of rhBSSL, said method comprising the steps of: providing a lyophilized formulation or a unit dose of the present invention; and reconstituting said lyophilized formulation or unit dose in a liquid. In a related aspect, the invention relates to a reconstituted formulation of rhBSSL, comprising: (i) said rhBSSL present in an absolute amount of between about 10 mg and about 20 mg; (ii) mannitol present in an absolute amount of between about 27 mg and about 62 mg; and (iii) glycine present in an absolute amount of between about 2 mg and about 6 mg; and wherein said formulation is reconstituted in a liquid infant feed and said reconstituted formulation has a pH of between about 6.4 and about 7.4.

In a further aspect, the invention relates to a use of glycine to stabilize rhBSSL, present in a lyophilized formulation further comprising a crystalline bulking agent that is not glycine, wherein: said glycine is present in said lyophilized formulation substantially in non-crystalline form; and/or said glycine is included in said lyophilized formulation at a relative amount of between about 0.2 mg and about 0.3 mg per mg of said rhBSSL.

In yet a further aspect, the invention relates to a method of reducing and/or minimizing the formation of insoluble aggregates of rhBSSL present in a liquid infant feed, said method comprising the steps of: providing a lyophilized formulation or a unit dose of the present invention; and reconstituting said lyophilized formulation or unit dose in a liquid infant feed.

In a particular aspect, the invention also relates to a method of determining a reduction in aggregation of rhBSSL, said method comprising the steps of: (i) providing a lyophilized formulation or a unit dose, of the present invention; (ii) storing said lyophilized formulation or unit dose; and (iii) determining, at one or more time-points, the percentage high molecule weight (% HMW) levels of said rhBSSL in said lyophilized formulation or said unit dose, thereby determining the level of aggregation of said rhBSSL.

### BRIEF DESCRIPTION OF THE DRAWINGS

FIG. 1 shows the instability of rhBSSL monomers (quantified by the integrated main peak detected by SE-HPLC) during storage at +5° C. between 0 and 18 months for the lyophilized formulations of experiment AH7507: (a) shows the reduction in the absolute % of integrated main peaks; and (b) shows the reduction in the relative % decrease (0 month main peak set as 100% for each formulation) of integrated main peaks. The general classes of concentration of glycine present in each pre-formulation is indicated by the shading of the plotted symbols, with solid symbols representing a “High” glycine concentration of 77 mM, the open symbols representing a “Low” glycine concentration of 0 mM and the hatched symbols representing “Medium” glycine concentrations of 27 mM (for N6 and N7) and 33 mM (for N4).

FIG. 2 shows the instability of rhBSSL monomers (quantified by the integrated main peak detected by SE-HPLC) during storage at +25° C. between 0 and 18 months for the lyophilized formulations of experiment AH7507: (a) shows the reduction in the absolute % of integrated main peaks; and (b) shows the reduction in the relative % decrease (0 month main peak set as 100% for each formulation) of integrated main peaks. The concentration of glycine present in each pre-formulation is indicated using the same shading as described above.

FIG. 3 shows the instability of rhBSSL monomers (quantified by the integrated main peak detected by SE-HPLC) during storage at +40° C. between 0 and 9 months for the lyophilized formulations of experiment AH7507: (a) shows the reduction in the absolute % of integrated main peaks; and (b) shows the reduction in the relative % decrease (0 month main peak set as 100% for each formulation) of integrated main peaks. The concentration of glycine present in each pre-formulation is indicated using the same shading as described above.

FIG. 4 shows the rate of rhBSSL aggregation (quantified by sum of all integrated high molecular weight peaks (HMW) detected by SE-HPLC) during storage at +5° C. between 0 and 18 months for the lyophilized formulations of experiment AH7507: (a) shows the rate using the absolute % of total integrated HMW peaks; and (b) shows the rate using the relative % increase (0 month total HMW peaks set as 100% for each formulation) of total integrated HMW peaks. The concentration of glycine present in each pre-formulation is indicated using the same shading as described above.

FIG. 5 shows the rate of rhBSSL aggregation (quantified by sum of all integrated high molecular weight peaks (HMW) detected by SE-HPLC) during storage at +25° C. between 0 and 18 months for the lyophilized formulations of experiment AH7507: (a) shows the rate using the absolute % of total integrated HMW peaks; and (b) shows the rate using the relative % increase (0 month total HMW peaks set as 100% for each formulation) of total integrated HMW peaks. The concentration of glycine present in each pre-formulation is indicated using the same shading as described above.

FIG. 6 shows the rate of rhBSSL aggregation (quantified by sum of all integrated high molecular weight peaks (HMW) detected by SE-HPLC) during storage at +40° C. between 0 and 9 months for the lyophilized formulations of experiment AH7507: (a) shows the rate using the absolute % of total integrated HMW peaks; and (b) shows the rate using the relative % increase (0 month total HMW peaks set as 100% for each formulation) of total integrated HMW peaks. The concentration of glycine present in each pre-formulation is indicated using the same shading as described above.

FIG. 7 shows an overlay of PXRD patterns obtained from N4, N5, N6 and N7 for the lyophilized formulations of experiment AH7507. The arrows mark the peaks corresponding to crystalline beta-glycine which are found to be present in formulation N5 only.

FIG. 8 shows a scatter plot representing the relationship between rhBSSL aggregation (quantified by sum of all integrated high molecular weight peaks (HMW) detected by SE-HPLC) against the concentration of glycine in the pre-lyophilized formulation for formulations of experiment AH7507 after storage at +40° C. for 9 months. The concentration of mannitol present in each pre-formulation is indicated by the shading of the plotted symbols, with solid squares representing a “High” mannitol concentration of 307 mM, the open squares representing a “Low” mannitol concentration of 132 mM and the hatched squares representing “Medium” mannitol concentration of 220 mM.

FIG. 9 shows a coefficient plot of the integrated main peak detected by SE-HPLC for the MLR model based on data from the formulations of experiment AH7507 after storage at +5° C. and +25° C. for 18 months.

FIG. 10 shows a contour surface from the MLR model used to analyze the integrated main peak detected by SE-HPLC (rhBSSL monomers) from the formulations of experiment AH7507 after storage at +5° C. for 18 months.

FIG. 11 shows a coefficient plot of the sum of all integrated high molecular weight peaks (HMW) detected by SE-HPLC (rhBSSL aggregates) for the MLR model based on data from the formulations of experiment AH7507 after storage at +5° C. and +25° C. for 18 months.

FIG. 12 shows a contour surface from the MLR model used to analyze the sum of all integrated high molecular weight peaks (HMW) detected by SE-HPLC (rhBSSL aggregates) from the formulations of experiment AH7507 after storage at +5° C. for 18 months.

FIG. 13 shows the instability of rhBSSL monomers—quantified by the integrated main peak detected by SE-HPLC—for the lyophilized formulations of experiments AH7513 and AH7517 after storage at +5° C. after storage for 0 to 12 months: (a) reduction in % of integrated main peak; and (b) relative % reduction (0 month main peak set as 100% for each formulation) of integrated main peak. Note that for experiment AH7517, data were collected at 0, 6, 9 and 12 months only. The general classes of concentration of glycine present in each pre-formulation is indicated by the shading of the plotted symbols, with solid symbols representing a “High” glycine concentration of 56 mM, the open symbols representing a “Low” glycine concentration of 0 mM and the hatched symbols representing “Medium” glycine concentrations of 44 mM (for G2) and 50 mM (for G3)

FIG. 14 shows the instability of rhBSSL monomers—quantified by the integrated main peak detected by SE-HPLC—for the lyophilized formulations of experiments AH7513 and AH7517 after storage at +25° C. for 0 to 12 months: (a) reduction in % of integrated main peak; and (b) relative % reduction (0 month main peak set as 100% for each formulation) of integrated main peak. Time points collected and the concentration of glycine present in each pre-formulation is indicated using the same shading as described above.

FIG. 15 shows the instability of rhBSSL monomers—quantified by the integrated main peak detected by SE-HPLC—for the lyophilized formulations of experiments AH7513 and AH7517 after storage at +40° C.: (a) reduction in % of integrated main peak after storage for 0 to 12 months. Note that for experiment AH7517, data were collected at 0, 3 and 6 months only; and (b) relative % reduction (0 month main peak set as 100% for each formulation) of integrated main peak after storage for 0 to 6 months. The concentration of glycine present in each pre-formulation is indicated using the same shading as described above.

FIG. 16 shows the rate of rhBSSL aggregation—quantified by the sum of all integrated high molecular weight peaks (HMW) detected by SE-HPLC—for the lyophilized formulations of experiments AH7513 and AH7517 after storage at +5° C. for 0 to 12 months: (a) increase of total integrated HMW peaks; and (b) relative % increase (0 month total HMW peaks set as 100% for each formulation) of total integrated HMW peaks. Note that for experiment AH7517, data were collected at 0, 6, 9 and 12 months only, and the concentration of glycine present in each pre-formulation is indicated using the same shading as described above.

FIG. 17 shows the rate of rhBSSL aggregation—quantified by the sum of all integrated high molecular weight peaks (HMW) detected by SE-HPLC—for the lyophilized formulations of experiments AH7513 and AH7517 after storage at +25° C. for 0 to 12 months: (a) increase of total integrated HMW peaks; and (b) relative % increase (0 month total HMW peaks set as 100% for each formulation) of total integrated HMW peaks. Time points collected and the concentration of glycine present in each pre-formulation is indicated using the same shading as described above.

FIG. 18 shows the rate of rhBSSL aggregation—quantified by the sum of all integrated high molecular weight peaks (HMW) detected by SE-HPLC—for the lyophilized formulations of experiments AH7513 and AH7517 after storage at +40° C.: (a) increase of total integrated HMW peaks after storage for 0 to 12 months. Note that for experiment AH7517, data were collected at 0, 3 and 6 months only; and (b) relative % increase (0 month total HMW peaks set as 100% for each formulation) of total integrated HMW peaks after storage for 0 to 6 months. The concentration of glycine present in each pre-formulation is indicated using the same shading as described above.

FIG. 19 shows PXRD patterns: obtained from: (a) the lyophilized formulation of rhBSSL F1 of experiment AH7513. The arrows mark the peaks corresponding to crystalline beta-glycine present in this formulation; and (b) the lyophilized formulation of rhBSSL G3 of experiment AH7517. The arrows mark the expected location of peaks (missing in this formulation) that would otherwise indicate the presence of crystalline beta-glycine.

FIG. 20 shows SDS-PAGE results for lyophilized formulations of rhBSSL stored for 12 months at various temperatures, and their respective degree of high molecular weight (HMW) aggregates for: (a) formulations F1 and F2 of experiment AH7513; and (b) formulations G2 and G3 of experiment AH7517.

### DISCLOSURE OF THE INVENTION

The present invention, and particular non-limiting aspects and/or embodiments thereof, can be generally described in more detail as follows:

In one aspect, the present invention relates to a formulation suitable for lyophilization comprising: (i) recombinant human bile salt-stimulated lipase (rhBSSL); (ii) a crystalline bulking agent; and (iii) an amorphous stabilizer that is a different chemical entity to said crystalline bulking agent.

Terms as set forth hereinafter are generally to be understood by their common meaning unless indicated otherwise.

Where the term “comprising” or “comprising of” is used in the present description and claims, it does not exclude other elements. For the purposes of the present invention, the term “consisting of” is considered to be a preferred embodiment of the term “comprising of”. If hereinafter a group is defined to comprise at least a certain number of embodiments, this is also to be understood to disclose a group which preferably consists of all and/or only of these embodiments.

In the context of the present invention, the terms “about” and “approximately” denote an interval of accuracy that the person skilled in the art will understand to still ensure the technical effect of the feature in question. The term typically indicates deviation from the indicated numerical value by ±20%, ±15%, ±10%, and preferably ±5%. As will be appreciated by the person of ordinary skill, the specific such deviation for a numerical value for a given technical effect will depend on the nature of the technical effect. For example, a natural or biological technical effect may generally have a larger such deviation than one for a man-made or engineering technical effect.

Where an indefinite or definite article is used when referring to a singular noun, e.g. “a”, “an” or “the”, this includes a plural of that noun unless something else is specifically stated.

I. Lyophilization in General

Lyophilization (also called freeze-drying) refers to a process that uses low temperature and pressure to remove a solvent, typically water, from a liquid formulation by the process of sublimation (i.e., a change in phase from solid to vapor without passing through a liquid phase). Lyophilization helps stabilize pharmaceutical formulations by reducing one or more solvent components to levels that no longer support chemical reactions or biological growth.

Freeze-drying processes are known. In some instances, freeze-drying is performed in a “manifold” process in which flasks, ampoules or vials are individually attached to the ports of a manifold or drying chamber. In other instances, freeze-drying is performed as a “batch” process in which one or more similar sized vessels containing like products are placed together in a tray dryer, hi a “bulk” process, the product is poured into a bulk pan and dried as a single unit. The product is removed from the freeze drying chamber prior to closure and then packaged in air-tight containers. The invention described herein can be used in combination with any of these processes.

Generally, lyophilization takes place in at least three stages: freezing; primary drying; and secondary drying. In some instances, it may be desirable to include an annealing step between the freezing and primary drying stages.

In the first step of a typical freeze-drying process, a sample of aqueous protein solution is cooled to below the product's collapse temperature until the solution is frozen. During the second step of primary drying, a vacuum is applied to the frozen material and in some cases heat is transferred to the frozen mass resulting in sublimation. Generally, freeze-drying is used to remove water from a solution or formulation. As sublimation occurs, water vapor passes from the frozen mass through to a freeze drying chamber. As the temperature increases, there is a higher saturated vapor pressure which results in an increased rate of drying. This results in a shortened freeze drying cycle. An upper limit on the drying temperature during this stage ensures that the temperature of the product is maintained below the product's collapse temperature.

“Collapse” of a product during freeze-drying is associated with a decreased surface area of dried formulation, reduction in volume and may also be associated with increasing the subsequent reconstitution time. In the event that the freeze-dried material collapses, solvent which has not been removed can become trapped. This may reduce undesirably the stability of the final product and have an adverse impact on its performance. The collapse temperature is the temperature at which the material softens to the point of not being able to support its own structure. In general, as the level of solvent is reduced via sublimation, the collapse temperature increases. In most systems which contain a protein, the onset of this temperature is not well defined and can occur over a range of temperatures. A material that will sustain this higher structural stability at a higher temperature may therefore allow faster processing. Components of the mixture may, therefore, impart stability during the freeze-drying process in addition to stabilizing the protein during subsequent storage.

In addition to the free ice that is sublimed during primary drying, there remains a substantial amount of water molecules that are bound to the product. In the third step of secondary drying, this is the water that is removed (desorbed). Since all of the free ice has been removed in primary drying, the product temperature can now be increased considerably without fear of collapse. Secondary drying (desorption) actually starts during the primary phase, but at elevated temperatures (typically in the 30° C. to 50° C. range), desorption proceeds much more quickly. Secondary drying rates are dependent on the product temperature. System vacuum may be continued at the same level used during primary drying, or may be varied. Secondary drying is continued until the product has acceptable moisture content for long term storage. Depending on the application, moisture content in fully dried products is typically between 0.5% and 3%.

Once dehydration by lyophilization is complete, the protein is left as a powder or “cake”. Lyophilization helps stabilize pharmaceutical formulations by reducing one or more solvent components (typically water) in the cake to levels that no longer support significant rates of chemical or physical degradation. The structure of the cake is important in allowing the material (e.g. the therapeutic protein and any other excipients) to be reconstituted. If the cake has small pores, the removal of water during the freeze-drying process can be impeded. As a result, the drying process is incomplete and the cake has a high moisture content. If the cake is formed with large pores, the drying process is more efficient and the cake has a low moisture content.

II. Formulations

As described above, lyophilization is a process in which a liquid formulation suitable for lyophilization is subjected to a freeze-dry process to obtain a lyophilized (freeze-dried) formulation. The contents of a freeze-dried formulation may vary depending upon the active agent and the intended route of administration. The liquid formulation generally includes a solvent and solute. The solute typically includes an active agent and, optionally, one or more excipients. The resulting freeze-dried formulation includes an amorphous solid matrix and a minor amount of residual unfrozen solvent. The amorphous solid matrix includes the active agent and, optionally, one or more excipients.

In general, any component in the formulation that is not the solvent or the active agent is referred to as an “excipient.” “Excipients” are included in a formulation for many reasons, although the primary function of many excipients is to provide a stable liquid environment for the active ingredient or to protect the active agent during the freezing or drying process. Some excipients may be used to achieve multiple effects in a formulation. For example, a disaccharide such as sucrose may act as a cryoprotectant, lyoprotectant, bulking agent and tonicity modifier. Behavior of an excipient may change when in the presence of other excipients. Some combinations have a positive synergistic effect, others have a negative synergistic effect. Positive synergy occurs when the sum of the effects of excipients acting together is greater than the additive effects of the individual excipients. Negative synergy occurs when the sum of effects of the combination of excipients is less than that of the individual excipients. Examples of active agents, solvents and excipients are provided below.

Active Agent and Recombinant Human Bile Salt-Stimulated Lipase:

As used herein, the term “pharmaceutical formulation” refers to both formulations that include at least one active agent, which is, or one of which is, recombinant human bile salt-stimulated lipase (rhBSSL).

Recombinant human bile salt-stimulated lipase (rhBSSL) as a component in the various aspects of the invention is the protein described, defined or referred to herein. For example, it includes polypeptides recognizable by a person of ordinary skill in the art as being human bile salt-stimulated lipase, wherein said human lipase has been produced by or isolated from a non-human source, such as a non-human organism, adapted or modified (for example by recombinant genetic technology) to produce such polypeptide.

Human bile salt-stimulated lipase (BSSL) is an enzyme known by various identifiers or aliases; for example, “carboxyl ester lipase (CEL)”, “bile salt-activated lipase (BAL)”, “bile salt-dependent lipase (BSDL)”, “carboxylesterase”, “carboxylic ester hydrolase” (CEH), and a number of other alias and descriptions as will be readily available to the person ordinarily skilled in the art from information sources such as “GeneCards” (www.genecards.org). A number of natural amino acid sequences and isoforms of human BSSL have been identified from human milk (and pancreas), and a number of different amino acid sequences (typically, predicted from cDNA or genomic sequence) have been described; all of which herein are encompassed within the term “human bile salt-stimulated lipase”. For example, human bile salt-stimulated lipase is naturally produced first as a precursor sequence including a 20 to 26 amino acid signal sequence, and the mature full-length form of the protein described as having 722 to 733 amino acids (for example see, Nilsson et al, 1990; WO 91/15234, WO 91/18923; the polypeptide predicted from cDNA sequence GenBank submission ID: X54457; GenBank ID: CAA38325.1; GeneCards entry for “CEL/BSSL”; GenBank ID: AAH42510.1; RefSeq ID: NP_{—}001798.2; Swiss-Prot ID: P19835). In further examples, other shorter isoforms of human bile salt-stimulated lipase are described in Venter et al (2001; Science, 291: 1304-1351); GenBnk ID: AAC71012.1; Pasqualini et al (1998; J Biol Chem, 273: 28208-28218); GenBank ID: EAW88031.1; WO 94/20610 and Bläckberg et al (1995; Eur J Biochem, 228: 817-821).

In particular embodiments, the human bile salt-stimulated lipase comprises a protein having an amino acid sequence comprising, or as shown by, SEQ ID NO: 2. In other particular embodiments, the (recombinant) human bile salt-stimulated lipase has an amino acid sequence of either the mature or precursor forms of BSSL selected from those disclosed in Nilsson et al, 1990; WO 91/15234, WO 91/18923; RefSeq ID: NP_{—}001798.2; GenBank ID: AAH42510.1; GenBank ID: CAA38325.1; GeneCards entry for “CEL/BSSL”; Swiss-Prot ID: P19835. In further such embodiments, the (recombinant) human bile salt-stimulated lipase comprises a protein with an amino acid sequence that is at least 720 consecutive amino acids of any of the sequences disclosed in the preceding references or of SEQ ID NO: 2. In other embodiments the (recombinant) human bile salt-stimulated lipase comprises a protein having at least the amino sequence from position 1 to 101 of that disclosed in SEQ ID NO: 2. or WO 91/15234, or at least the amino acid sequence from position 1 to 535 of that disclosed in SEQ ID NO: 2, such as “Variant A” disclosed in Hansson et al, 1993; J Biol Chem, 35: 26692-26698, wherein such protein has bile salt-binding and/or bile salt-dependent lipase activity, as for example may be determined by the methods disclosed in Bläckberg et al (1995; Eur J Biochem 228: 817-821).

It will now therefore be apparent to the person ordinarily skilled in the art that in certain embodiments of the present invention one or more of these described forms of (recombinant) human bile salt-stimulated lipase may be useful in the various aspects of the invention. Further, it will be apparent to such person that other (recombinant) proteins that have bile salt-dependent lipolytic activity (for example, as may be determined by the methods disclosed in Bläckberg et al, 1995) and that are similar in amino acid sequence to those polypeptide sequences described, defined or referred to herein may also have utility in the present invention, and hence are also encompassed by the term “human bile salt-stimulated lipase”. In certain such embodiments, a protein that shows more than 90%, 95%, 98%, 99%, 99.5% sequence identity over at least about 30, 50, 100, 250, 500, 600, 700, 711, 720, 722, 733 or 750 amino acids to a sequence described, defined or referred to herein. In other embodiments, one or more amino acid substitutions may be made to one of the BSSL polypeptide sequences disclosed, defined or referred to herein. For example, one, two, three, four, five or up to 10 amino acid substitutions, deletions or additions may be made to the sequence disclosed in SEQ ID NO: 2. Such amino acid changes may be neutral changes (such as neutral amino acid substitutions), and/or they may affect the glycosylation, binding, catalytic activity or other properties of the protein in some (desired) manner. Proteins with such substitutions, providing they have bile salt-dependent lipolytic activity, will also be recognized by the person ordinarily skilled in the art as being “human bile salt-stimulated lipase” in the sense of the present invention.

In other embodiments the human bile salt-stimulated lipase is expressible from or otherwise encoded by a nucleic acid having a suitable nucleic acid sequence. By way of non-limited example, said lipase is expressible from or otherwise encoded by a nucleic acid comprising the sequence between positions 151 and 2316 of SEQ ID NO: 1, or that disclosed in WO 94/20610 or Nilsson et al (1990). As will also be appreciated by the person of ordinary skill, a “suitable nucleic acid sequence” will also encompass variants of the preceding nucleic acid sequences. For example, changes in one or more nucleotide bases that do not change the amino acid encoded by a triplet-codon (such as in the 3^{rd} codon position) will also be “suitable”. Sub-fragments of such nucleic acid sequences will also be “suitable” if they encode a (short) isoform of human bile salt-stimulated lipase as described herein. Furthermore, nucleic acid sequences that encode a protein having a variant of the amino acid sequence shown by SEQ ID NO: 2, such as those described above, will also be “suitable”. Accordingly, the present invention envisions embodiments whereby the (recombinant) human bile salt-stimulated lipase is a protein that is expressible or otherwise encoded by a nucleic acid that hybridizes to a nucleic acid comprising the sequence between positions 151 and 2316 of SEQ ID NO: 1 or to one comprising the sequence between positions 151 and 755, and wherein said protein has bile salt-dependent lipolytic activity. In certain such embodiments, the hybridization is conducted at stringent conditions, such as will be known to the person of ordinary skill, and is described in general text books for example “Molecular Cloning: A Laboratory Manual”, by Joe Sambrook and David Russell (CSHL Press).

In a particular embodiment, the (recombinant) human bile salt-stimulated lipase is produced by expression from a nucleic acid described, defined or referred to herein.

A human bile salt-stimulated lipase described, defined or referred to herein, in the context of the present invention is a recombinant bile salt-stimulated lipase (rhBSSL); i.e. where said human lipase has been produced by or isolated from a non-human source, such as a non-human organism, adapted or modified (for example by recombinant genetic technology) to produce such lipase. In particular embodiments, the rhBSSL is produced using cell-free and/or in-vitro transcription-translation techniques from an isolated nucleic acid molecule described, defined or referred to herein. Alternatively, a recombinant non-human organism is used, wherein said non-human organism includes at least one copy of such a nucleic acid, and where said nucleic acid is expressible by said non-human organism to produce the desired protein: rhBSSL. For example, recombinant bacterial, algae, yeast or other eukaryotic cells may be used, and the rhBSSL is, in certain embodiments, produced from the culture of such recombinant cells. In other embodiments, the rhBSSL may be produced by extra-corporal culture of modified or specifically selected human cells, for example by their in-vitro culture. In yet other embodiments, rhBSSL may be produced by its isolation from the milk of transgenic animals; such as transgenic cattle, sheep, goats or rabbits. The person or ordinary skill in the art will be aware of the numerous technologies available to produce human bile salt-stimulated lipase using recombinant technology.

Recombinant human bile salt-stimulated lipase has been shown to be producible from recombinant cell culture including the culture of E. coli, mouse and hamster (Hansson et al, 1993), and P. pastoris (Trimple et al, 2004; Glycobiol, 14: 265-274) cells. Recombinant human bile salt-stimulated lipase has also been shown to be producible and isolatable from the milk of transgenic mice (Strömqvist et al, 1996; Transgen Res, 5: 475-485) and from the milk of transgenic sheep (WO 99/54443). In certain embodiments of the present invention, the recombinant human bile salt-stimulated lipase is isolated from the culture of such recombinant cells or from the milk of such transgenic animals. In an alternative embodiment, the recombinant human bile salt-stimulated lipase is not one isolated from the milk of a transgenic sheep or a transgenic mouse.

In a particular embodiment of the present invention, the recombinant human bile salt-stimulated lipase is isolated from an expression product of a recombinant Chinese hamster ovary (CHO) cell line, is produced by a recombinant CHO cell line, or is expressible by, or isolatable from, a recombinant CHO cell line. Use of a recombinant CHO cell line expression system to produce such lipase can produce rhBSSL that exhibits particular structural, activity or other characteristic features, such as one or more of those described in co-pending applications WO 2012/052059 and WO 2012/052060, the contents of which are incorporated herein by reference. By way of non-limiting example, the rhBSSL useful in the present invention may be isolated using a process and/or exhibit characteristics analogous to, or substantially as described in, the Exemplification herein, or as described in co-pending applications WO 2012/052059 and WO 2012/052060.

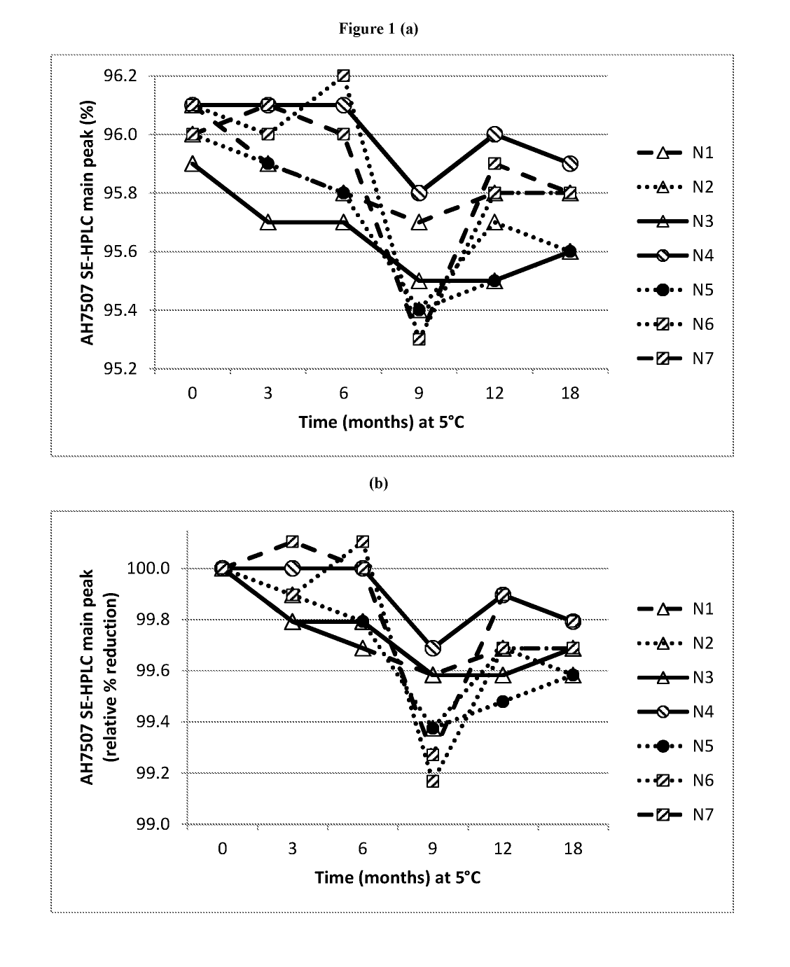

In certain embodiments of the present invention, the recombinant human bile salt-stimulated lipase is identified by the International Non-proprietary Name (INN) stem “bucelipase” (see WHO Drug Information, 21: 62, 2007), for example because it has the amino acid sequence shown therein. The recombinant human bile salt-stimulated lipase, when used in the present invention may, with reference to SEQ ID NO: 2, have one or more disulfide bridges at the locations Cys64-Cys80 and Cys246-Cys257, and/or is glycosylated at one or more of the possible glycosylation sites at Asn-187, Thr-538, Thr-549, Thr-559, Thr-576, Thr-587, Thr-598, Thr-609, Thr-620, Thr-631 and Thr-642 (in one such embodiment, schematically represented in FIG. 1.1). In certain such embodiments, the rhBSSL is in a glycoform, and may for example, have the INN of “bucelipase alfa”.

In other particular embodiments of the present invention, the recombinant human bile salt-stimulated lipase has structural, composition and/or other properties that are different to those of native human bile salt-stimulated lipase (BSSL-MAM) and/or different from that form of recombinant bile salt-stimulated lipase that has been produced by isolation from the milk of transgenic sheep (rhBSSL-OVI), such as described in WO 99/54443. Certain of such structural and/or composition differences, or other properties that are different, are described in co-pending applications WO 2012/052059 and WO 2012/052060. By way of non-limiting example, in certain such embodiments, the recombinant human bile salt-stimulated lipase useful for the present invention is (substantially) free of other milk proteins or milk components. As will be apparent upon the disclosure of the present invention, in certain embodiments the rhBSSL is added to a milk-based infant feed before administration to the human infant Accordingly, in such embodiments, the “free of other milk proteins or milk components” will apply to that form, composition or formulation of the recombinant bile salt-stimulated lipase that exists shortly before (such as immediately before) addition of said lipase to said milk-based infant food. For example, in such embodiments the pharmaceutical compositions or kits components of the invention containing rhBSSL, or that amount of rhBSSL that is provided ready for addition to any infant formula and/or pasteurized breast milk, are free of such milk-based contaminates. In certain such embodiments, the rhBSSL is free of milk casein and whey proteins, such as lactoferrin, or free of other contaminates native to milk, in particular where such milk-derived proteins or other contaminates are derived from the milk of humans, sheep or mice. In these embodiments, the “free of” any particular such protein or contaminant means that no material amounts of such protein or other contaminate can be detected by routine detection methodologies. Alternatively, any such particular impurity may be present at a level of less than about 5%, such as less than about 2%, 1%, 0.5% or 0.1%, or is essentially or effectively absent, or that the total of all such milk-derived proteins or other contaminates are present at a level of less than about 5%, such as less than about 2%, 1%, 0.5% or 0.1%, or are essentially or effectively absent. As will be understood by the person ordinarily skilled in the art, recombinant human bile salt-stimulated lipase produced & isolated from cell culture, such as from recombinant CHO cells will be considered “free of” such milk-based contaminates.

In other certain such embodiments of the present invention, the recombinant human bile salt-stimulated lipase has purity of greater than about 70%, such as a purity of greater than about 80%, 90% or 95%. In particular such embodiments, such percentage purity is a percentage purity of total protein. As described above, in the applicable embodiments such purity measure is that of the composition comprising said lipase before addition to any infant feed or other administration medium. Such purity values may be determined by RP-HPLC, SE-HPLC or SDS-PAGE (with SyproRuby or silver staining) techniques.

In other embodiments of the invention, particularly if the recombinant human bile salt-stimulated lipase is produced using (expressed from) recombinant CHO cells, the rhBSSL when used in the present invention may be characterized by one or more structural, activity or other properties such as those described in the following. Methods to determine such structural, activity or other properties will be known to the person of ordinary skill upon the disclosure of the present invention and, for example, include those described in co-pending applications WO 2012/052059 and WO 2012/052060.

In further certain such embodiments of the invention, the recombinant human bile salt-stimulated lipase has a level (overall/total) of glycosylation that is less than that of native human bile salt-stimulated lipase (BSSL-MAM) and/or has a level (overall/total) of glycosylation that is more than that of recombinant human bile salt-stimulated lipase isolated from the milk of transgenic sheep (rhBSSL-OVI). The levels of glycosylation, such as the level of monosaccharide and/or sialic acid content of BSSL (or sample thereof) may be measured using high pH anion exchange chromatography with pulsed amperiometric detection (HPAEC-PAD). In particular embodiments of the present invention, the total monosaccharide content of the recombinant human bile salt-stimulated lipase (moles monosaccharide per mole rhBSSL) is between about 20 and 100, between about 25 and 65 or between about 25 and 55, such as between about 40 to 45 mole/(mole rhBSSL), In certain embodiments of the invention the total sialic acid content of the rhBSSL (moles sialic acid per mole rhBSSL) is between about 20 and 35, such as between about 25 and 30 mole/(mole rhBSSL).

In yet other certain such embodiments of the present invention, the recombinant human bile salt-stimulated lipase has a glycosylation pattern, for example of 0-glycans, that is different to that of BSSL-MAM and/or different to that of rhBSSL-OVI. Such differences may be detected using capillary electrophoresis with laser induced fluorescence detection (CE-LIF) and/or HPAEC-PAD. In particular embodiments of the invention, the rhBSSL may have between about 20 and 50 mole of N-acetyl neuraminic acid (NANA=Neu5Ac) per mole rhBSSL [mole/(mole rhBSSL)], such as between about 25 and 40 mole/(mole rhBSSL). The rhBSSL used in the invention may have less than about 5 mole N-glycosyl neuraminic acid (NGNA=Neu5Gc) per mole rhBSSL, such as less than about 2 mole/(mole rhBSSL), or where NGNA is essentially undetectable. The rhBSSL used in the invention may have less than about 20 mole fucose per mole rhBSSL, such as less than about 10, less than about 5, less than or about 2 mole/(mole rhBSSL), and in certain embodiments fucose is essentially undetectable. The rhBSSL used in the invention may have between about 5 and 25 mole galactosamine per mole rhBSSL, such as between about 10 and 20 or between about 15 and 18 mole/(mole rhBSSL). The rhBSSL used in the invention may have less than about 10 mole glucosamine per mole rhBSSL, such as less than about 5, less than about 3 or about 2 mole/(mole rhBSSL). The rhBSSL used in the invention may have between about 5 and 25 mole galactose per mole rhBSSL, such as between about 10 and 20 or between about 15 and 18 mole/(mole rhBSSL). The rhBSSL used in the invention may have less than about 5 mole glucose per mole rhBSSL, such as less than about 2 mole/(mole rhBSSL), or where glucose is essentially undetectable. The rhBSSL used in the invention may have between about 2 and 8 mole mannose per mole rhBSSL, such as between about 4 and 6 mole/(mole rhBSSL). In particular embodiments of the invention, the rhBSSL may have a profile of monosaccaride and/or sialic acid content about that as, or substantially as, represented in Table 1.1 of co-pending applications WO 2012/052059 and WO 2012/052060.

In other embodiments of the invention, the recombinant human bile salt-stimulated lipase useful for the present invention is different from BSSL-MAM and from rhBSSL-OVI in the profile or amount of lectin binding or Lewis-antigen binding tests, such as those assays and profiles described in Bläckberg et al (1995) and Landberg et al (1997) respectively. Such lectin binding or Lewis-antigen binding tests can indicate differences in glycosylation pattern between these different forms of BSSL. Other techniques may be used to identify and/or characterize recombinant human bile salt-stimulated lipase useful for the present invention. For example, rhBSSL may be characterized (and/or differentiated from BSSL-MAM or from rhBSSL-OVI) by endoprotease Lys-C digestion followed by analysis of the resulting peptides with reverse-phase HPLC with quantitative UV detection (at 214 nm), and recording/inspection of the resulting chromatogram. Differences in the resulting chromatogram may be due to—and hence further reflect—unique features of glycosylation of specific peptides comprising the rhBSSL that have specific differences in retention time.

In yet further embodiments of the present invention, the recombinant human bile salt-stimulated lipase has a molecular mass of between 90 kDa and 75 kDa. In particular such embodiments the molecular mass of said lipase is between about 84 and 86 kDa, such as about 85 kDa. The molecular mass may be determined by routine techniques including MALDI-MS. By way of comparison, using the same detection techniques the molecular mass of BSSL-MAM is measured as being substantially greater (for example, around 100 kDa) and that of rhBSSL-OVI is measured as being substantially smaller (for example, around 78 kDa).

In other further such embodiments of the present invention, the recombinant human bile salt-stimulated lipase can comprise a population of recombinant human bile salt-stimulated lipase molecules having sequences of different amino acid lengths. In certain of such embodiments, the amount of lipase molecules that are present in a form that is shorter at the C-terminal end by one, two, three, four, five or up to ten amino acids, compared to the longest or (predicted) full-length form (such as that shown by SEQ ID NO: 2) is greater than 50% of the amount of lipase molecules present in such longest or (predicted) full-length form. In certain such embodiments, between about 100% and 500% of the amount of the longest (or predicted full-length) lipase molecule is the amount present as a shorter lipase molecule, such as by one or two amino acids from the C-terminal end. In particular such embodiments between about 200% and 400%, for example about 300%, of the amount of the longest (or predicted full-length) molecule (for example, that shown by SEQ ID NO: 2), is the amount present as a shorter lipase molecule such as by one or two amino acids from the C-terminal end. In particular embodiments or the foregoing, less than 1% of the amount of the longest (or predicted full length) said lipase molecules is present as a lipase molecule shorter by two amino acids. In other embodiments, between two- to five-fold, such that about three-fold, the number of longest (or predicted) said lipase molecules are present in a form that are shorter than such longest (or predicted) molecule from the C-terminal end by one, two, three, four, five or up to ten amino acids.

In yet other further such embodiments of the present invention, the recombinant human bile salt-stimulated lipase may have a specific activity that is greater than BSSL isolated from human milk and/or rhBSSL-OVI. For example, the specific activity of the rhBSSL may be between about 15% and 35% higher, such as about 20% or 25% higher specific activity than that of BSSL-MAM and/or rhBSSL-OVI (based on mass). Techniques to measure specific activity of human BSSL will be known to the person of ordinary skill and include using the 4-nitrophenyl ester butyric acid (PNPB) assay as generally described in the Exemplification herein. Other in-vitro assays for BSSL are known, for example by use of trioleoylglycerol emulsified in gum Arabic as the substrate for BSSL and sodium cholate (10 mM) as activating bile salt (for example, as described by Bläckberg and Hernell, 1981; Eur J Biochem, 116: 221-225). In particular embodiments, prior to measuring specific activity, the BSSL may be purified to a high purity, such as by using the techniques of heparin-affinity chromatography and size exclusion chromatography.

As will be understood by the person of ordinary skill, the recombinant human bile salt-stimulated lipase used in the present invention may be characterized by more than one of the distinguishing features described or defined herein, such as those above. For example, a combination of two or more (such as three, four, five or more) of such features may together characterize a particular embodiment of the recombinant human bile salt-stimulated lipase for use in the present invention.

In certain embodiment of this aspect of the invention, said rhBSSL is present at a concentration of between about 1 mg/mL and about 35 mg/mL; preferably wherein said concentration is between about 10 mg/mL and about 15 mg/mL; more preferably wherein said concentration is selected from the group consisting of about: 11 mg/mL; 12 mg/mL; 13 mg/mL; and 14 mg/mL. In alternative embodiments, such as those where a lower effective amount of the rhBSSL is desired, said rhBSSL is present at a concentration of about between about 1 and about 5 mg/mL; preferably about 2, 3 or 4 mg/mL.

As will be now within the ability of the person or ordinary skill, the amount/concentration of rhBSSL present in a formulation or composition may be expressed in absolute amount (e.g. mass or molar quantities) and/or in terms of the number of active units. The activity of rhBSSL may be easily determined using the PNPB assay as described herein, with reference to an active standard BSSL molecule. Suitable masses of active rhBSSL are within the ranges of masses given above. As the molecular mass of a complex protein such as rhBSSL may vary, for example due to differences in glycosylation, the amount of said lipase may be defined in ways other than in terms of mass, such as in terms of (active) molar amounts. The skilled person will be readily able to make other conversions from specific mg amounts to the corresponding micro mole amount. Alternatively, the amount of recombinant human bile salt-stimulated lipase may be expressed in terms of the activity of the lipase in enzyme units (U), such as defined as the amount of said lipase that catalyzes the formation of 1 micro mole of product per minute under the conditions of the assay, for example as determined in an in vitro assay for BSSL activity such as one described herein.

Solvent

As discussed previously, lyophilization is the process by which solvent is removed from a liquid formulation. As used herein, the term “solvent” refers to the liquid component of a formulation that is capable of dissolving or suspending one or more solutes. The term “solvent” can refer to a single solvent or a mixture of solvents. A commonly used solvent for pharmaceutical formulations is water for injection (WFI). Depending on the formulation or the freeze-drying process, it may be desirable to include one or more organic solvents in the liquid formulation. For example, it may be desirable to include an organic solvent in the formulation to enhance the solubility of one or more active ingredients. Examples of suitable organic solvents include, but are not limited to, acetonitrile, methanol, ethanol, propanol, tert-butyl alcohol, acetone, cyclohexane, and dimethylsulfoxide (DMSO).

Bulking Agent

The purpose of the bulking agent is to provide bulk to the formulation and enhance cake formation. As used herein, the term “bulking agent” includes both “crystalline” and “non-crystalline” bulking agents. The term “crystalline” bulking agents refer to bulking agents that are capable of forming a crystal structure under typical lyophilization conditions.

In general, a crystalline bulking agent refers to a bulking agent that is capable of crystallizing during freezing (for example, between a temperature of about 0° C. and about −50° C.). A crystalline bulking agent may require an annealing, thermal treatment step or other component to promote crystallization during the freezing process. For example, a bulking agent may or may not crystallize during lyophilization, depending upon the conditions of the lyophilization process and/or the other excipients present in the formulation. Typically, when a sufficient amount of crystalline bulking agent is included in a liquid formulation (e.g., when the ratio of crystalline bulking agent to amorphous solute/component (for example, rhBSSL or other excipients) is at least about 1.0, about 1.25 or about 1.5) and allowed to crystallize during the lyophilization process, the crystalline bulking agent may form a structural support matrix for the amorphous component(s) of the formulation (for example, rhBSSL. As used herein, the term “structural support matrix” refers to the support that the crystalline structure provides to the formulation (analogous to a “scaffolding”), such that the macrostructure of the cake is largely unaffected by any “microcollapse” of the amorphous solute residing within the interstices of the structural support matrix during primary drying. This crystalline structural support matrix may allow for primary drying with a product temperature above the glass transition temperature of the amorphous component(s) of the product.

In certain embodiments of his aspect, the relative mass-concentration of crystalline bulking agent to rhBSSL is greater than about 1 to 1, such as greater than about 1.25 to 1, greater than about 1.5 to 1, greater than about 2.0 to 1 or greater than about 2.5 to 1, such as between about 2.0 to 1 and 5.0 to 1.

In particular embodiments of all aspects of the invention, said crystalline bulking agent is not an amino acid, such as a polyol, for example, where said crystalline bulking agent is mannitol.

As evidenced by Example 3, the inventors surprisingly find that the addition of a crystalline bulking agent, such as mannitol, significantly improves the stability of a lyophilized formulation of rhBSSL compared to a liquid formulation that does not include a crystalline bulking agent.

In particular embodiments of the formulation suitable for lyophilization of the present invention, said crystalline bulking agent is mannitol, present at a concentration of between about 50 mM and about 500 mM; preferably wherein said concentration is about between 100 mM and 400 mM, or between 150 mM and 300 mM. In preferred such embodiments, the concentration of mannitol is about between about 175 mM and about 250 mM, and more preferably wherein said mannitol is present at a concentration of between about 180 mM and about 210 mM; such as wherein the concentration of mannitol is selected from the group consisting of about: 185 mM; 190 mM; 195 mM; 200 mM; and 205 mM.

Stabilizing Agents

Stabilizing agents can comprise the formulations and/or compositions of the present invention. In particular embodiments the formulations and/or compositions of the invention may further comprise a stabilizing agent, such as an amorphous stabilizer, that is a different chemical entity to said crystalline bulking agent.

The term “amorphous” stabilizer refers to stabilizing agents that are capable of taking an amorphous form under typical lyophilization conditions. The term “amorphous” is commonly understood by the person of ordinary skill, and includes the meaning to describe a solid that lacks—to a detectable degree—the long-range order characteristic of a crystal.

In certain embodiments of the formulations and/or compositions of the present invention, the amorphous stabilizer is not sucrose; preferably said amorphous stabilizer is not a saccharide; more preferably said amorphous stabilizer is an amino acid. In particular such embodiments, said amorphous stabilizer is selected from the group consisting of: L-arginine; L-histidine; L-proline; L-alanine; and glycine; most preferably wherein said amorphous stabilizer is glycine.

As evidenced by the examples herein, the inventors surprisingly find that the addition of an amorphous stabilizing agent, such as glycine, has an additional and synergistic effect on the stability of rhBSSL present in the lyophilized formulation. Such advantageous effects are shown, in particular, when the glycine is present in the formulation within certain ranges of concentrations/amounts.

Accordingly, in particular embodiments of the formulation suitable for lyophilization of the present invention, said amorphous stabilizer is glycine, present in such formulation at a concentration of between about 10 mM and about 100 mM; preferably wherein said concentration of glycine is between about 20 mM and about 70 mM; more preferably wherein said concentration is about between 30 mM and 55 mM; most preferably said glycine is present in such formulation at a concentration is about between 35 mM and 50 mM, such as at a glycine concentration selected from the group consisting of about: 36 mM; 38 mM; 40 mM; 42 mM; 44 mM; 46 mM; and 48 mM.

pH or Buffering Agents

Buffers are typically included in pharmaceutical formulations to maintain the pH of the formulation at a physiologically acceptable pH. The desirable pH for a formulation may also be affected by the active agent. For example, most biopharmaceutical active agents have a higher activity within a specific pH range. Generally, the pH of the formulation is maintained between about 4.0 and about 8.0, between about 5.5 and about 7.5, or between about 6.0 and about 7.2. Typically the buffer is included in the liquid formulation at a concentration between about 2 mM to about 50 mM, or between about 10 mM and 25 mM.

Examples of suitable buffers include buffers derived from an acid such as phosphate, aconitic, citric, gluaric, malic, succinic and carbonic acid. Typically, the buffer is employed as an alkali or alkaline earth salt of one of these acids. Frequently the buffer is phosphate or citrate, often citrate, for example sodium citrate or citric acid. Other suitable buffers include acetate, Tris and histidine buffers.

In particular embodiments of the formulation suitable for lyophilization, the formulation has a pH value of between about 6.3 and about 7.5; preferably said pH value is between about 6.6 and about 7.2; more preferably wherein said pH value is selected from the group consisting of about: 6.7; 6.8; 6.9; 7.0; and 7.1.

In certain embodiments, the formulation suitable for lyophilization can further comprise a sodium phosphate buffer. In particular such embodiments, the formulation suitable for lyophilization comprises sodium phosphate, present at a phosphate concentration of between about 2 mM and about 20 mM; preferably wherein said phosphate concentration is between about 5 mM and about 15 mM; more preferably wherein said phosphate concentration is selected from the group consisting of about: 6 mM; 8 mM; 10 mM; 12 mM; and 14 mM.

It will be understood by a person of ordinary skill that a concentration of phosphate will encompass any or the three forms of phosphate forms (H₃PO₄, (H₂PO₄)⁻, (HPO₄)²⁻ or (PO₄)³⁻) at applicable relative concentrations, depending on the pH, which at biological pH ranges will typically comprise (H₂PO₄), (HPO₄)²⁻ ions as the predominate phosphate form. Accordingly, at physiological pHs, a sodium phosphate buffer is typically provided by an equilibrium between disodium hydrogen phosphate and sodium dihydrogen phosphate.

Other Excipients

Other excipients may be added to any of the formulations/compositions of the present invention. Other excipients may include isotonic agents such as salts, and/or preservatives, sweeteners, colorings, fillers, etc,

In particular embodiments, the formulations/compositions of the present invention may further comprises sodium chloride. For example, the formulation suitable for lyophilization may comprise sodium chloride, present at a chloride concentration of about between 10 mM and 30 mM; preferably wherein said chloride concentration is about 15 mM and 25 mM; more preferably wherein said chloride concentration is selected from the group consisting of about: 18 mM; 20 mM; 22 mM; and 24 mM.

III. Specific Formulations and Other Aspects of the Present Invention

The inventors disclose herein a particular formulation including rhBSSL that is suitable for lyophilization having a specific combination of excipients within a specific range of concentration. As evidenced in the examples, such a formulation has particular utility in forming a lyophilized formulation of rhBSSL that shows improvements in one or more characteristics has described herein. Accordingly, in a particular embodiment, a formulation suitable for lyophilization of the present inventions comprises:
rhBSSL present at a concentration of about between 10 mg/mL and 15 mg/mL;
mannitol present at a concentration of about between 180 mM and 210 mM;
glycine present at a concentration of about between 35 mM and 50 mM;
sodium phosphate present at a phosphate concentration of about between 2 mM and 20 mM, preferably about between 5 mM and 15 mM; and
sodium chloride present at a chloride concentration of about between 5 mM and 50 mM, preferably about between 15 mM and 25 mM,
wherein the formulation has a pH value of about between 6.3 and 7.2.

Upon freeze-drying of such a (liquid) formulation, such as by a method as described herein, a lyophilized formulation of rhBSSL is formed that shows improvements in one or more characteristics has described herein.

Accordingly, another aspect of the present invention relates to a lyophilized formulation obtainable by, such as is obtained from, lyophilization of a formulation suitable for lyophilization, as described herein.

In certain of such aspects, the rhBSSL in said lyophilized formulation is present substantially in non-crystalline form. For example, less than about 20%, 10%, 5%, 2%, 1%, 0.5% or 0.1% of said rhBSSL may be in crystalline form, or no crystalline form of rhBSSL may be detectable, e.g. by powder X-ray diffraction analysis. In preferred such embodiments, said rhBSSL is present in amorphous form.

In particular embodiments of the lyophilized formulation, the crystalline bulking agent is mannitol. In certain of such embodiments, said mannitol is present substantially in crystalline form; and/or said mannitol is included at a relative amount of between about 1 mg and about 10 mg per mg of said rhBSSL. Preferably, mannitol is included at a relative amount of between about 2 mg and about 5 mg, more preferably between about 2.7 mg and about 3.1 mg, per mg of rhBSSL. By “present substantially” with reference to a component means that between about 5% and about 50%, such as between about 10% and about 50% or between about 25% and about 50% of the component is in the given form. In preferred embodiments, said mannitol is present predominately in crystalline form. By “present predominately” with reference to a component means that more than about 50%, such as more than about 60%, 70%, 80%, 90% or 95% of the component is in the given form.

The inventors demonstrate that other than the crystalline bulking agent, surprisingly no other crystalline form was detected in certain of the lyophilized formulations of the present invention. In particular, and with reference to the formulation described in Example 4, there was no evidence of crystalline glycine, crystalline sodium phosphate and even no evidence of crystalline sodium chloride.

Accordingly, in preferred embodiments of the lyophilized formulation, said mannitol is the only component of said formulation that is present substantially in crystalline form. For example, mannitol is the only component within the lyophilized formulation for which crystals can be detected; such as by detection using powder X-ray diffraction analysis. In alternative embodiments of the lyophilized formulation, said mannitol is the only excipient that is present substantially in crystalline form; or wherein said mannitol is the only bulking agent present in the formulation; and/or is the only bulking agent present in crystalline form, such as present substantially in crystalline form.

The lyophilized formulation of the present invention comprises an amorphous stabilizer that is a different chemical entity to said crystalline bulking agent. In preferred such embodiments of the lyophilized formulation of the present invention, said amorphous stabilizer is glycine, and: said glycine is present substantially in non-crystalline form; and/or said glycine is included at a relative amount of between about 0.1 mg and about 0.5 mg per mg of said rhBSSL. Preferably, glycine is included at a relative amount of between about 0.1 mg and about 0.4 mg, more preferably between about 0.2 mg and about 0.3 mg, per mg of rhBSSL. In more preferred embodiments, the lyophilized formulation of the present invention comprises, as said amorphous stabilizer, glycine, wherein said glycine present in amorphous form. For example, no glycine can be detected in crystalline form by powder X-ray diffraction analysis, such as particularly by the absence of detectable peaks characteristic of crystalline glycine for example the absence of detectable powder X-ray diffraction peaks at D-values 17.906, 23.693 and/or 28.429 2θ.

In particular such preferred forms of the lyophilized formulation, said glycine is the only stabilizer present in the formulation, and/or is the only stabilizer present in substantially non-crystalline form, and in yet more preferred forms glycine is the only stabilizer present in amorphous form.

In further embodiments, the lyophilized formulation of the present invention comprises sodium phosphate; preferably wherein said sodium phosphate is present substantially in non-crystalline form; and/or said sodium phosphate is included at a relative amount of between about 0.015 mg and about 0.25 mg per mg of said rhBSSL. In such embodiments, said sodium phosphate may comprise disodium hydrogen phosphate and sodium dihydrogen phosphate.

The inventors demonstrate the surprising finding that despite the presence of glycine in certain of the formulations of the invention (an excipient known to promote crystallization of sodium phosphate), crystalline sodium phosphate is not detectable. Accordingly, in certain embodiments, the lyophilized formulation of the present invention comprises sodium phosphate present in amorphous form.

In yet further embodiments, the lyophilized formulation of the present invention comprises sodium chloride; preferably wherein said sodium chloride is present substantially in non-crystalline form; and/or said sodium chloride is included at a relative amount of between about 0.02 mg and about 0.3 mg per mg of said rhBSSL.

The inventors demonstrate the surprising finding that despite sodium chloride normally forms crystals readily, crystalline sodium phosphate is not detectable. Accordingly, in certain embodiments, the lyophilized formulation of the present invention comprises sodium chloride is present in amorphous form.

The inventors disclose herein a particular lyophilized formulation including rhBSSL having a specific combination of excipients within a specific range of relative amounts. As evidenced in the examples, such a lyophilized formulation shows improvements in one or more characteristics has described herein. Accordingly, in a particular embodiment, a lyophilized formulation of the present inventions comprises per mg of said rhBSSL:
mannitol, present at a relative amount of between about between about 2 mg and about 5 mg, preferably between about 2.7 mg and about 3.1 mg;
glycine, present at a relative amount of between about 0.1 mg and about 0.4 mg, preferably between about 0.2 mg and about 0.3 mg;
sodium phosphate, present at a relative amount of between about 0.05 mg and about 0.15 mg; and
sodium chloride, present at a relative amount of between about 0.06 mg and about 0.18 mg.

In preferred embodiments of such particular lyophilized formulation of the invention: mannitol is present substantially in crystalline form, preferable the mannitol is present predominately in crystalline form; glycine is present in amorphous form; sodium phosphate is present in amorphous form; and/or sodium chloride is present in amorphous form.

As will now be apparent to the person of ordinary skill, the lyophilized formulations of the present invention may be prepared in varying absolute amounts, such as in large manufacturing batches preparing, for example about: 100 g, 1 Kg, 10 Kg, 100 Kg, 250 Kg or 500 Kg of such lyophilized formulation. For administration to individuals such as patients in need, however, smaller amounts will be desired in amounts that may be administered, in singular or multiple such amounts, to the individual in any given administration or course of administrations.

Accordingly, in another aspect the invention relates to such a desired amount of a lyophilized formulation of the present invention, being a unit dose of a lyophilized formulation as described herein wherein said rhBSSL is present in such unit dose in an absolute amount of between about 1 mg and about 500 mg. In a preferred embodiment of such unit dose, the rhBSSL is present in an absolute amount of between about 5 mg and about 25 mg, and more preferably wherein said amount is selected from the group consisting of about: 8 mg; 10 mg; 12 mg; 14 mg; and 16 mg. By way of non-limiting examples of applications for such unit doses, those unit doses comprising between about 25 mg and about 50 mg may have utility in treating adult cystic fibrosis and/or pancreatic insufficiency patients; and those unit doses comprising between about 10 mg and about 20 mg may have utility in treating infants such as pre-term infants. In certain embodiments, such as where a “half-dose” may be required to supplement a whole unit dose, for example when accurate dose to body weight is required such as for administration to pre-term and/or small infants, a unit dose of the present invention may comprise rhBSSL present in an absolute amount of between about 2 mg and 10 mg, such as an amount of rhBSSL selected from about: 4 mg; 6 mg; and 8 mg. As described herein, the person of ordinary skill will now readily be able to represent the amount of rhBSSL in any lyophilized formulation or unit dose of the present invention in terms of an amount of active rhBSSL such as by a number of enzyme units (U) by, for example, using an activity assay as described herein.

In certain embodiments, a unit dose of the present invention is useful for, and/or is specifically adapted for, administration to pre-term infants For example, said administration can include administration of a liquid infant feed via the gastrointestinal tract, where said unit dose of said lyophilized formulation has been reconstituted into said infant feed prior to said administration. In certain embodiments, the liquid infant feed is a milk-based or fat-based (such as milk- or vegetable-fat based) liquid infant feed. In particular embodiments, the liquid infant feed is pasteurized breast milk, and in alternative embodiments it is an infant formula such as one disclosed in co-pending applications WO 2012/052059 and WO 2012/052060. Administration via the gastrointestinal tract can be conveniently conducted by feeding, such by bottle. Alternatively, the administration may be effected by other means; for example, by use of a dropper, syringe, spoon or a soaked-cloth, such as may be required if the infant has a deformity of the mouth. In certain embodiments, such as with extremely underweight, preterm or weak infants, the administration may be made directly to the gastrointestinal tract via a gastric, gastrostomy, or duodenal tube.

Pre-term infants are particularly at risk, and hence require careful feeding and administration of therapeutic agents. Accordingly, there is a great need for therapeutic agents for administration to such infants that are stable, and hence retain their therapeutic effect over long periods of time. Significant or substantial changes to the stability of therapeutic agents for administration to such infants may lead to incomplete-, over- or variable-dosing to such infants during a course of therapy; potentially with deadly results.

Accordingly, in certain embodiments the present invention includes the lyophilized formulation as described herein, or the unit dose as described herein, wherein said rhBSSL comprises stable rhBSSL. By “stable” is meant that the therapeutic activity or potential of the rhBSSL, and/or the formulation as a whole, is maintained for the desired period of time upon storage at a recommended dosage. By way of non-limiting example, such desired time period may be for at least about: 3 months, 6 months, 12 months, 12, months, 18 months, 24 months or longer, such as 72 months: and such recommend storage temperature may be about −18° C., +4° C., +18° C. or about +22° C.

In certain embodiments of the invention, the lyophilized formulation or the unit dose comprising stable rhBSSL does not readily form aggregates, such as during storage for such periods and time periods. In certain of such embodiments, the aggregates are insoluble aggregates. The presence of insoluble aggregates of rhBSSL in a therapeutic formulation, even one given orally and particularly one given to pre-term infants, may have significant effects on dosage and hence efficacy and/or safety of the therapy. A reduction in the amount of insoluble aggregates of rhBSSL would therefore be desired as it may contribute to less variation in efficacy and safer therapeutic uses of rhBSSL. By way of non-limiting example, “does not readily form” aggregates includes that after storage at +25° C. for 6 months, less than about 5%, such as less than about 3%, 2.5% or 2% of rhBSSL aggregates are present. The amount of rhBSSL aggregates can be quantified, for example, by SE-HPLC as described herein. Alternatively, the rate of aggregate formation may be less than that shown for formulation N1 described herein.

In certain embodiments, the formation of aggregates in said lyophilized formulation or unit dose is the result of storage of said lyophilized formulation at a temperature of between about 0° C. and about +40° C.; preferably wherein said storage temperature is selected from the group consisting of about: +5° C.; +10° C.; +15° C.; +20° C.; and +25° C. In other embodiments, such formation of aggregation may result from surface interactions, (UV) light, radiation, chemical modification, presence of surfactants.

In certain embodiments, the shelf-life of the lyophilized formulation or the unit dose is prolonged, for example to up to about 18 months, 25 months, or 72 months, upon storage at +4° C., +18° C. or about +22° C.

In yet another aspect, the invention relates to a method of producing a lyophilized formulation of rhBSSL, said method comprising the steps of: providing a formulation suitable for lyophilization as described, defined or claimed herein; and lyophilizing said formulation. Said method may comprise the steps of: freezing said formulation suitable for lyophilization; primary drying said frozen formulation; and secondary drying the primary dried formulation.

In preferred embodiments of this method, each steps of such method may be conducted using parameters as described or defined for such step herein. For example, and as set out in more detail in Example 4 herein, the step of freezing may be conducted by cooling the formulation suitable for lyophilization to about −50° C. at a rate of about 0.8° C./hour, and further such embodiments the frozen formulation may be equilibrated by maintaining at −50° C. for 5 hours. With respect to primary drying, such step may be conducted by applying a vacuum of about 0.2 mbar with a shelf temperature of 0° C., and continued for about 13 hours and/or until the temperature of the sample approached that of the shelf indicating that sublimation of ice crystals is complete. Secondary drying may be initiated by lowering the chamber pressure to about 0.02 mbar and raising the temperature of the shelves to about +25° C. at a rate of about 1° C./hour, and secondary drying can be continued for about 10 hours until the product has a moisture content of between about 0.8% and 0.2%. Lyophilization of the liquid formulation may be conducted within glass vials placed in a lyophilization chamber; which vials are then, when lyophilization is compete, sealed under vacuum with rubber stoppers.

In a related aspect, the present invention also relates to a lyophilized formulation of rhBSSL obtainable by, such as obtained from, the method described above.

In order to administer the rhBSSL to an individual, the lyophilized formulations of the present invention are typically reconstituted; that is dissolved in a solvent (usually aqueous-based) to form a solution of rhBSSL that may be more readily bioavailable to said individual. Accordingly, in a further aspect the present invention relates to a method of producing a reconstituted formulation of rhBSSL, said method comprising the steps of: providing either a lyophilized formulation as described, defined or claimed herein, or a unit dose as described, defined or claimed herein; and reconstituting said lyophilized formulation or unit dose in a liquid, for example in a solvent such as an aqueous-based solvent.

In particular such methods, the resulting reconstituted formulation has a pH of about between 5.9 and 7.9; preferably wherein said pH is about between 6.4 and 7.4; more preferably wherein said pH is selected from the group consisting of about: 6.5; 6.6; 6.7; 6.8; 6.9; 7.0; 7.1; 7.2; and 7.3.

In further such methods, the resulting reconstituted formulation comprises said rhBSSL is in an amount of between about 2.5 mg and about 50 mg; preferably wherein said amount is between about 5 mg and about 25 mg; more preferably wherein said amount is selected from the group consisting of about: 7.5; 10; 12.5; 15, 17.5 and 20 mg.

Certain application of the present invention relates to the provision of formulations of rhBSSL suitable for administration to human infants. Accordingly, in certain embodiments of this method, the lyophilized formulation or unit dose is reconstituted in a liquid infant feed, and hence said constituted formulation is reconstituted in a liquid infant feed.

In certain embodiments of the present invention, the liquid infant feed is non-fresh breast milk into which the lyophilized formulation or unit dose is reconstituted is pasteurized breast milk. In other embodiments the breast milk has been frozen, such as after pasteurization. In particular embodiments, the breast milk used in the present invention has come from a breast milk bank. Breast milk banks may include the National Milk Bank (NMB), a nationwide organization that collects donated human milk, ensures milk safety and quality and makes it available for infants in need, or the Human Milk Banking Association of North America (HMBANA), a non-profit association of donor human milk banks established in 1985 to set standards for and to facilitate establishment and operation of milk banks in North America.

In an alternative embodiment, the lyophilized formulation or unit dose is reconstituted in an infant formula. The skilled person will be aware of the many infant formulae that are commercially available, which include: Enfamil™, Pregestimil™, Nutramigen™, and Nutramigen AA™ (all marketed or made by Mead Johnson); Similac™, Isomil™ Alimentum™, and EleCare™ (all marketed or made by Abbott Laboratories, Ross division); Nestlé: the largest producer of formula in the world, makes GoodStart™ (marketed or made by Nestle/Gerber Products Company); Farex1™ and Farex2™ (marketed or made by Wockhardt Nutrition). For preterm infants, other infant formulae such as Similac Neosure, Entramil Premature, Similac Special Care, Cow & Gate Nutriprem 2 and Entramil Enfacare are also available Common to all infant formula is that they contain a source of lipids that are the substrates to lipases such as rhBSSL. In a particular embodiment, the infant formula has the composition (before addition of rhBSSL) generally in conformance with, or substantially as the specifications shown in Exhibit A of co-pending applications WO 2012/052059 and WO 2012/052060, or as one recommended by the ESPGHAN Coordinated International Expert Group (Koletzko et al, 2005; J Ped Gastro Nutr 41: 584-599). In certain embodiments, the infant formula contains one or more of the ingredients, and at approximately the levels, shown in said Exhibit B. In particularly advantageous embodiments, the infant formula contains at least 0.5% (of total fat) that is docosahexaenoic acid (DHA) and/or arachidonic acid (AA), and in further such embodiments where the concentration of AA should reach at least the concentration of DHA, and/or if eicosapentaenonic acid (C20:5 n-3) is added its concentration does not exceed the content of DHA.

Such a liquid infant feed comprising reconstituted rhBSSL from the lyophilized formulation or unit dose of the present invention stored, for example, for 9 months at +25° C., is expected to have lower levels of insoluble aggregates than a liquid infant feed made from prior art rhBSSL formulations (an aqueous solution of rhBSSL), or made from lyophilized rhBSSL without any bulking or stabilizing agents, in each case similarly stored for 9 months at +25° C.

In a particular aspect, the present invention relates to a reconstituted formulation of rhBSSL, comprising:
(i) said rhBSSL present in an absolute amount of between 10 mg and 20 mg;
(ii) mannitol present in an absolute amount of between 27 mg and 62 mg;
(iii) glycine present in an absolute amount of between 2 mg and 6 mg; and wherein said formulation is reconstituted in a liquid infant feed; and said reconstituted formulation has a pH of between 6.4 and 7.4.

The inventors further demonstrate herein, that use of glycine in lyophilized formulations of rhBSSL provides further surprising and advantageous properties to such formulation of this specific protein. Accordingly, in another aspect, the present invention relates to a use of glycine to stabilize rhBSSL, present in a lyophilized formulation, wherein:
said glycine is present in said lyophilized formulation substantially in non-crystalline form; and/or
said glycine is included in said lyophilized formulation at a relative amount of between about 0.1 mg and about 0.4 mg, preferably between about 0.2 mg and about 0.3 mg per mg of said rhBSSL.

The said lyophilized formulation further comprises a crystalline bulking agent that is not glycine, such as a crystalline bulking agent being mannitol. In preferred embodiments of such aspect, said glycine present in said lyophilized formulation is present in amorphous form.

In more preferred such embodiments of such use, the lyophilized formulation further comprises, per mg of said rhBSSL:
mannitol, included in said lyophilized formulation as said crystalline bulking agent, at a relative amount of between about 2 mg and about 5 mg, preferably between about 2.7 mg and about 3.1 mg;
sodium phosphate is included in said lyophilized formulation at a relative amount of between about 0.05 mg and about 0.15 mg; and
sodium chloride is included in said lyophilized formulation at a relative amount of between about 0.06 mg and about 0.18 mg.

In yet further preferred such embodiments of such use, in such lyophilized formulation:
said mannitol is present substantially in crystalline form;
said glycine is present in amorphous form;
said sodium phosphate is present in amorphous form; and
said sodium chloride is present in amorphous form.

Such uses of glycine provide, in certain embodiments, formulations of rhBSSL having increased stability. Accordingly, in particular embodiments said stabilization of said rhBSSL is characterized by the rate of formation of aggregates of said rhBSSL. For example, said formation of aggregates is the result of storage of said lyophilized formulation at a temperature of between about 0° C. and +40° C.; preferably wherein said storage temperature is selected from the group consisting of about: +5° C.; +10° C.; +15° C.; +20° C.; and +25° C.

Accordingly, in a related aspect the present invention relate to a method of reducing and/or minimizing the formation of insoluble aggregates of rhBSSL present in a liquid infant feed, said method comprising the steps of: providing: a lyophilized formulation or a unit dose as described, defined or claimed herein; and reconstituting said lyophilized formulation or unit dose in a liquid infant feed.

In certain embodiments of such method, the formulation or unit dose is directly added to the liquid infant feed and dissolving it therein. In alterative embodiments, the lyophilized formulation or unit dose is first dissolved in a first liquid (such as water), which is then added to the liquid infant feed.

In other embodiments of such method, such method is practiced: to increase the amount of active rhBSSL present in solution in said liquid infant feed relative to the amount of insoluble aggregates; and/or to reduce variability in potency of the rhBSSL between different liquid infant feeds.

One important method in the characterization of the formulations and/or compositions of the present invention is the determination of the degree of rhBSSL aggregations. Accordingly, one further aspect of the present invention relates to a method of determining a reduction in aggregation of rhBSSL, said method comprising the steps of: providing a lyophilized formulation o or a unit dose as described, defined or claimed herein; storing said lyophilized formulation or unit dose; and determining, at one or more time-points, the percentage high molecule weight (% HMW) levels of said rhBSSL in said lyophilized formulation or said unit dose, thereby determining the level of aggregation of said rhBSSL. The degree and/or level of rhBSSL aggregation may be determined and/or quantified by any suitable method, such as by SE-HPLC to detect % HMW levels of rhBSSL, as for example described in the examples herein.

In preferred such methods, said method comprises the step of determining if said lyophilized formulation comprises less that about 3.5%, 3.0%, 2.5%, 2.25%, 2.0%, 1.75% or 1.5% HMW species of said rhBSSL, as determined by SE-HPLC, after storage at +5° C. for 18 months.

It is to be understood that application of the teachings of the present invention to a specific problem or environment will be within the capabilities of one having ordinary skill in the art in light of the teachings contained herein. All references, patents, and publications cited herein are hereby incorporated by reference in their entirety. Examples of the formulations and compositions of the present invention and representative methods, uses or processes for their preparation or use appear in the following.

### EXAMPLES

The following exemplification, including the experiments conducted and results achieved, also illustrate various presently particular embodiments of the present invention, and are provided for illustrative purposes only and are not to be construed as limiting the present invention.

### Example 1

### Experiment AH7507

Experimental set-up: The effect of a crystallizing bulking agent, and optionally an amorphous stabilizing agent, on the properties of a lyophilized formulation of rhBSSL was studied using a full factorial 2 level design (2²) with 2 centre points. Excipients in various combinations and amounts were used to produce 7 lyophilized formulations having the compositions presented in Table 1. Temperature was added as a third factor (storage for 18 months at +5° C. and +25° C.) to the factorial design for samples to be stored for 18 months, and for a shorter period of 9 months samples were also stored at +40° C. At regular periods during storage, samples were taken from the various lyophilized formulations, and studied using size-exclusion high-performance liquid chromatography (SE-HPLC), powder X-ray diffraction (PXRD) and other techniques.

TABLE 1
Amount of rhBSSL and excipients in lyophilized powder of the
lyophilized formulations of AH7507.

| Sam- | | | Sodium | Sodium | | |
|---|---|---|---|---|---|---|
| ple | rhBSSL enzyme | | Chloride | Phosphate* | Mannitol | Glycine |
| no. | mg/vial | U/vial | mg/vial | mg/vial | mg/vial | mg/vial |
| N1 | 15.0 | 8685 | 1.83 | 1.7 | 30 | 0.00 |
| N2 | 15.0 | 8685 | 1.83 | 1.7 | 50 | 0.00 |
| N3 | 15.0 | 8685 | 1.83 | 1.7 | 70 | 0.00 |
| N4 | 15.0 | 8685 | 1.83 | 1.7 | 30 | 3.09 |
| N5 | 15.0 | 8685 | 1.83 | 1.7 | 70 | 7.21 |
| N6 | 15.0 | 8685 | 1.83 | 1.7 | 50 | 2.58 |
| N7 | 15.0 | 8685 | 1.83 | 1.7 | 50 | 2.58 |

*Amount of sodium phosphate calculated from weighted-average molecular weights and mass of material of the two sodium phosphate components.

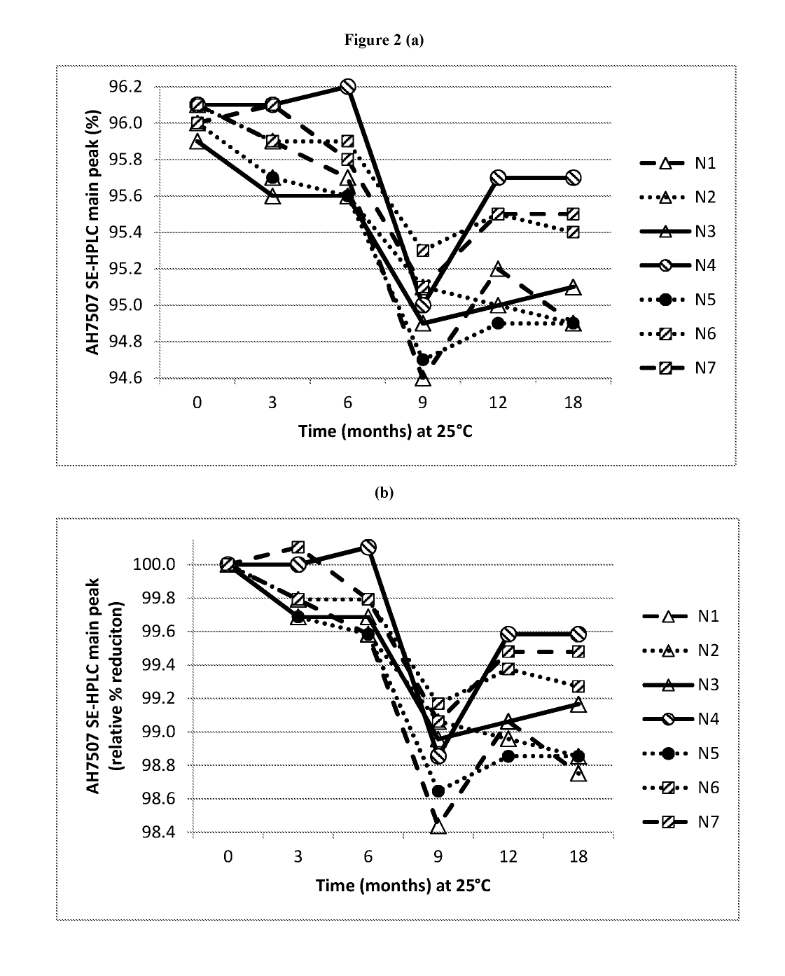

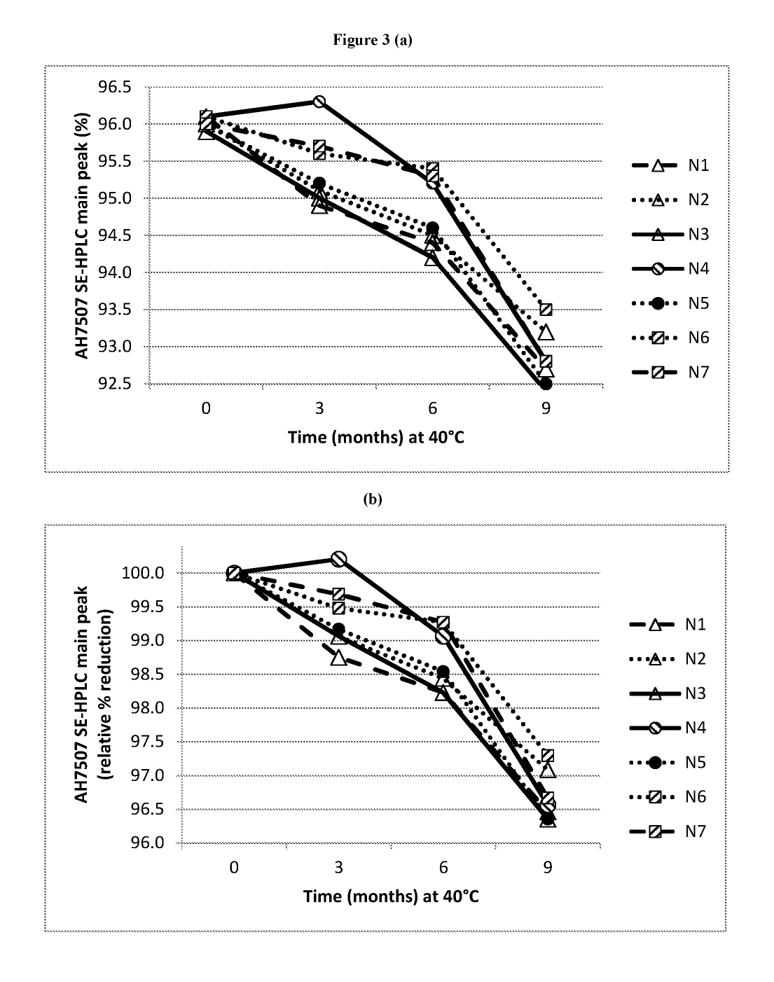

Results (i) monomerization and aggregation of rhBSSL studied by SE-HPLC: During long term storage, rhBSSL in monomeric form was detected as main peak and the formation of rhBSSL aggregates detected as higher molecular-weight peaks were studied using size-exclusion high-performance liquid chromatography (SE-HPLC). The two responses are connected as aggregation or rhBSSL would, by consequence, lower the remaining amount of rhBSSL monomer. FIG. 1 shows the amount of rhBSSL monomer (quantified by the main peak of SE-HPLC) present in the various lyophilized formulations after storage at +5° C. for various periods of time, FIG. 2 shows the same after storage at +25° C., and FIG. 3 shows the same after storage at +40° C.

Surprisingly, not only is the percentage of rhBSSL monomers higher for all formulations tested that contain a crystallizing bulking agent (in this example mannitol) than another formulation of rhBSSL (see Example 3): bulk lyophilized rhBSSL without any bulking or stabilizing agents, and/or an aqueous solution of rhBSSL, but that inspection of the results represented here show that in particular the lyophilized formulations N4, N6 and N7, which comprise an amount of glycine greater than 0 mg/vial but less than 7.21 mg/vial, have a reduced rate of loss of rhBSSL monomer compared to the other formulations that comprise either no glycine or 7.21 mg glycine per vial. This effect is observed most clearly after about 12 months of storage at +25° C., but evidence is also seen after the same duration of storage at +5° C., and between about 3 and 6 months of storage at the most extreme conditions of +40° C.

In FIGS. 1 to 6, the general classes of concentration of glycine that was present in each liquid formulation prior to lyophilization is also indicated by the shading of the plotted symbols, with solid symbols representing a “High” glycine concentration of 77 mM, the open symbols representing a “Low” glycine concentration of 0 mM and the hatched symbols representing

“Medium” glycine concentrations of 27 mM (for N6 and N7) and 33 mM (for N4). This coding, particularly at the +40° C. temperature, aids the interpretation of these graphs with respect to the greater stability of the formulations that were obtained from the “Medium” glycine concentrations.

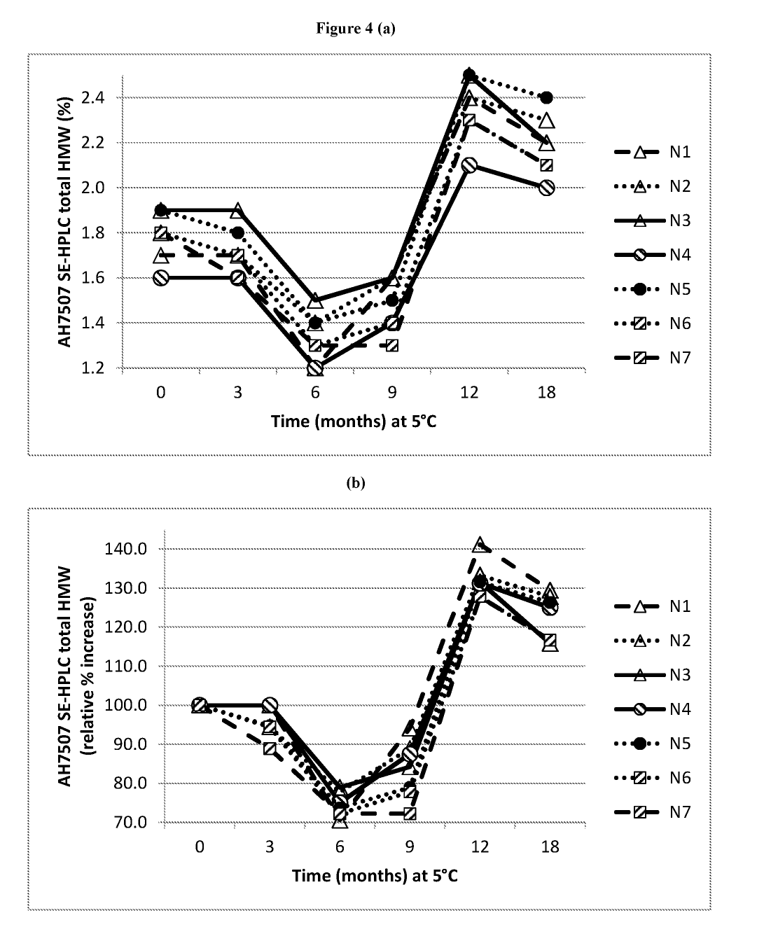

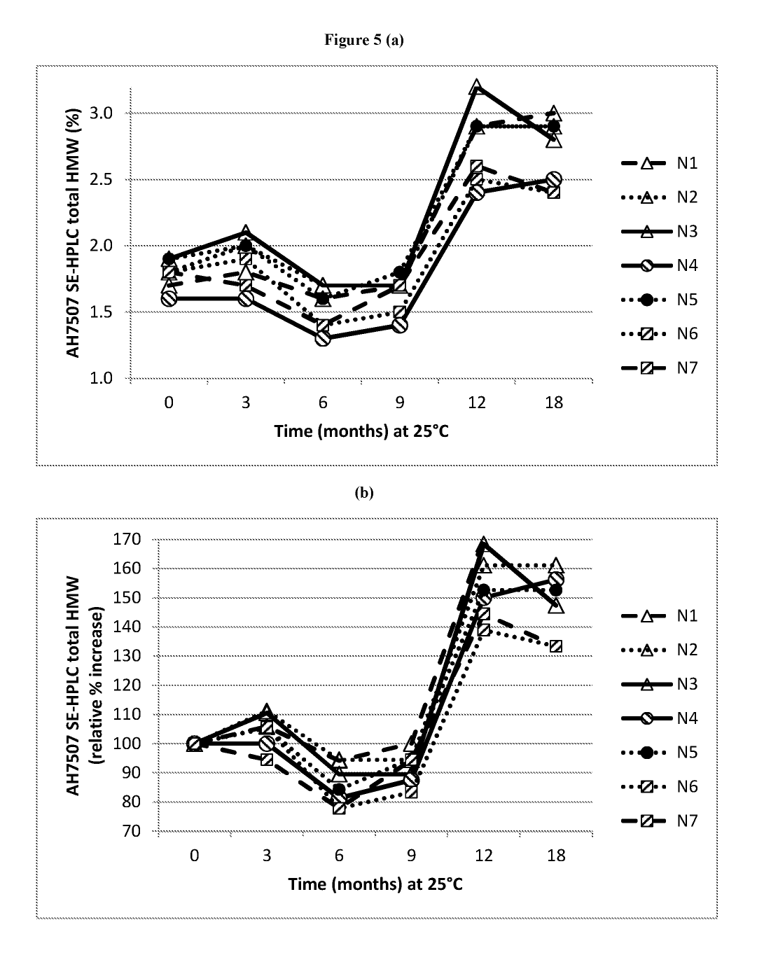

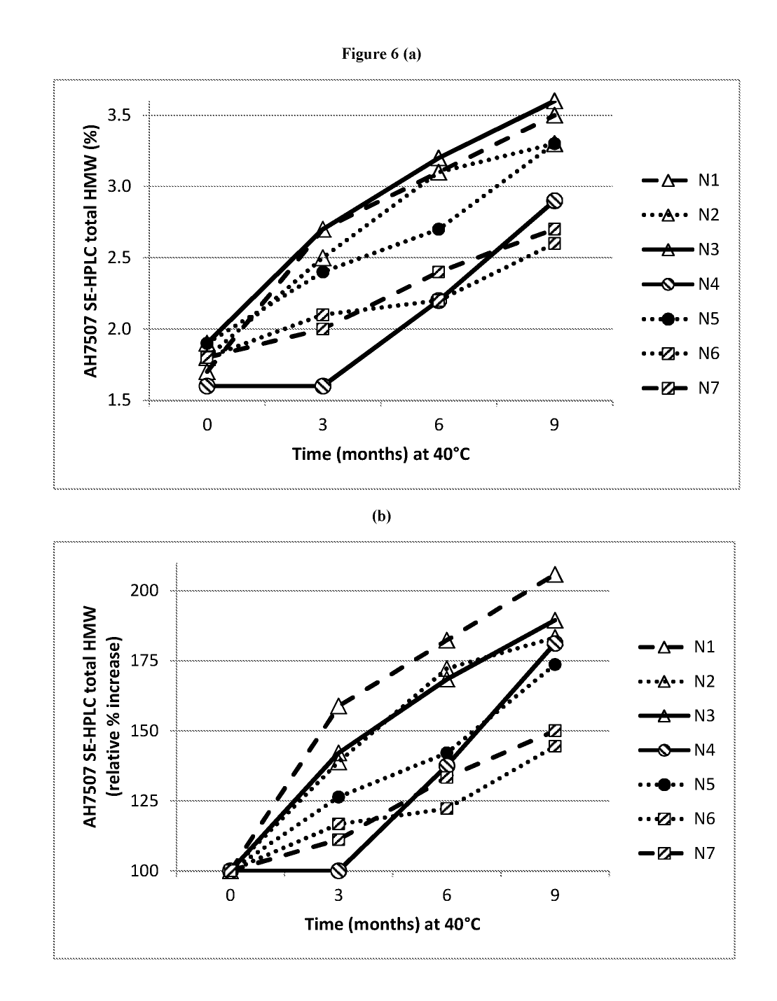

This surprisingly additional stabilizing effect of glycine, especially within a particular range of amounts, is additionally found supported from a lower rate of formation of rhBSSL aggregates for formulations N4, N6 and N7 compared to the other formulations, following storage at the three temperatures studied. FIG. 4 shows the amount of rhBSSL aggregates (quantified by the high molecular weight mains peak of SE-HPLC) present in the various lyophilized formulations after storage at +5° C. for various periods of time, FIG. 5 shows the same after storage at +25° C., and FIG. 6 shows the same after storage at +40° C. The effect is clearly seen at the higher storage temperatures of +25° C. and +40° C.

Results (n) insoluble aggregates of rhBSSL: Of significance is found that the aggregates of rhBSSL are not readily solubilized, for example upon agitation in a buffer containing 0.1% SDS.

Results (iii) crystallization of components of the formulation studied by PXRD: Powder X-ray diffraction (PXRD) was used to determine the crystalline form of the components of each formulation of experiment AH7507 at various time-points during the experiment, from time zero up to the final storage sampling. All sampled time-points for a given formulation gave similar results, that PXRD showed that the crystalline matrix in the lyophilized samples N1 to N7 were mainly appeared delta-mannitol, with some small amount of appeared beta mannitol detected in formulation N4. No evidence of crystalline rhBSSL was detected by PXRD.

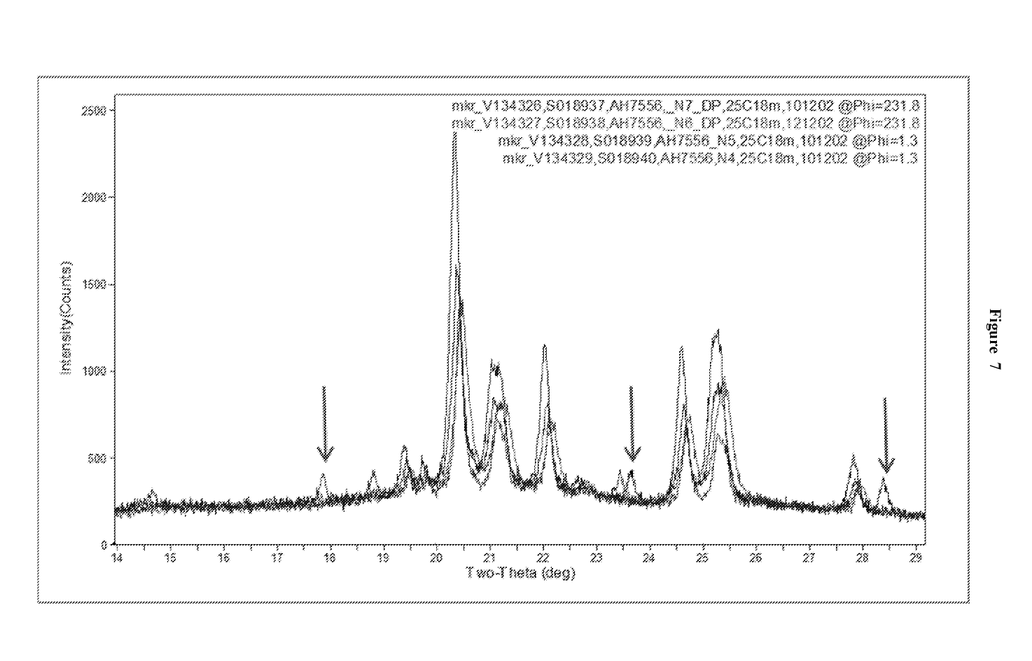

Surprisingly, despite prior art teaching that glycine readily crystallizes during freeze-drying, no crystalline glycine could be detected in formulations N4, N6 or N7; those formulations that from the SE-HPLC studies above appeared to show improved stability in terms of reduced loss of rhBSSL monomers and reduced formation of (insoluble) rhBSSL aggregates. In formulation N5 however, the formulation having the highest amount of glycine present (7.21 mg/vial), crystalline beta-glycine could be detected. In FIG. 7 the PXRD pattern for formulations N4, N5, N6 and N7 have been overlaid for comparison. The peaks indicating the presence of beta-glycine in formulation N5, but surprisingly not in any other of the glycine-containing formulations, have been marked with arrows.

Further surprisingly, no evidence of crystalline sodium chloride or sodium phosphate was detected in any of the formulations N4 to N7, despite both salts generally being believed to readily form crystals during freeze-drying.

Results (iv) Multiple linear regression (MLR) analysis: Data obtained from the formulations sampled at the last time points of storage were evaluated by MLR analysis in Modde 9.0 (Umetrics AB). The analysis was performed on the 9 month time point stored at +40° C. and the 18 month time point for the samples stored at +5° C. and +25° C. Samples from storage at +40° C. for 9 months were evaluated based on specific enzyme activity, SE-HPLC main peak (rhBSSL monomers) and HMW peaks (rhBSSL aggregates). Samples stored at +5° C. and +25° C. for 18 months were evaluated by MLR, with temperature added to a model in which samples from both temperatures were evaluated in the same model.

For the MLR model applied to data from samples stored at +40° C., in an evaluation of rhBSSL monomer (SEC-HPLC main peak), a small effect of mannitol was observed: the square effect of mannitol showed a negative effect to the SE-HPLC main peak (i.e., an increase in the square of the amount of mannitol is weakly associated with a reduction in the amount of rhBSSL monomers). This negative effect was low and just separated from the 95% confidence interval. None of the investigated factors showed any significant effect on the specific enzyme activity.

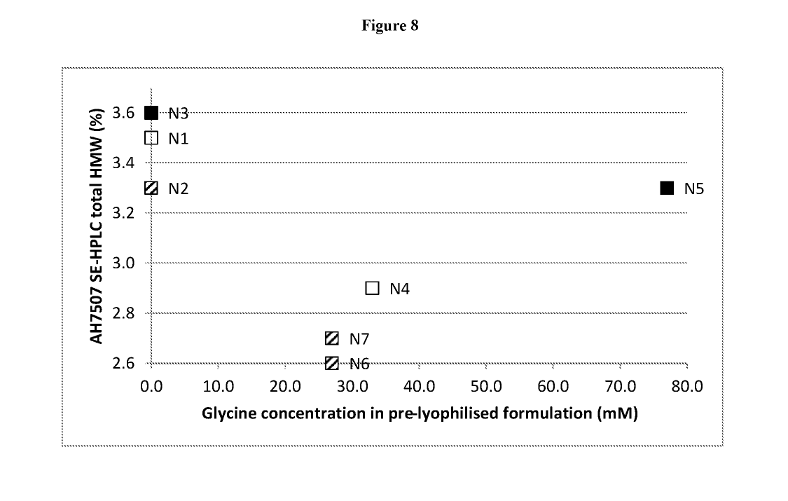

For the MLR model applied to data from samples stored at +40° C., in an evaluation of rhBSSL aggregates (SEC-HPLC HMW peaks), a significant effect of glycine was observed: the presence and amount of glycine accounted for a large portion of the variance (R² 0.991) in SE-HPLC HMW. Whilst Q² (cross validated R²) was relatively low at −10.032, from this analysis of a single time-point, this result is supported by the trend seen in FIG. 6, with glycine appearing to have the most effective stabilizing effect in an intermediate amount between zero and 7.21 mg/vial. This is more clearly seen in FIG. 8, which shows that the least advantageous formulations are those with complete absence of glycine (N1, N2 & N3—“category 1”) as these are the formulations with the highest percentage of SE-HPLC HMW. Almost as high percentage of SE-HPLC HMW is seen in sample N5 (category 1) indicating an unfavorable formulation in this sample as well. In the formulations with an intermediate amount of glycine (N4, N6 & N7—“category 2”) the percentage of SE-HPLC HMW is significantly lower. These category 2 samples are those that comprise glycine, but showed no evidence in PXRD of the presence of crystalline beta-glycine. These data indicate that the presence of glycine, in particular the presence of glycine within particular amounts and/or in non-crystalline form, has an effect of inhibiting formation/lowering the amount of aggregates in the freeze-dried samples. Interpretation of these data presented using the relative % increase in total HMW peaks, suggests that not only is an intermediate glycine concentration advantageous to reduce formation of rhBSSL aggregates, but that (by comparison of N6/N7 to N4) that the presence of some mannitol has a further synergistic effect by reducing the amount of (insoluble) rhBSSL aggregates after storage at +40° C. for 9 months.

The MLR model applied to samples stored at +40° C. was used to design a composition of one favorable formulation with respect to the lowest amounts of SE-HPLC HMW, which was determined by the model to consist of 4 mg glycine per vial and approximately 50 mg mannitol per vial (with the same amounts/ratios of salts and rhBSSL).

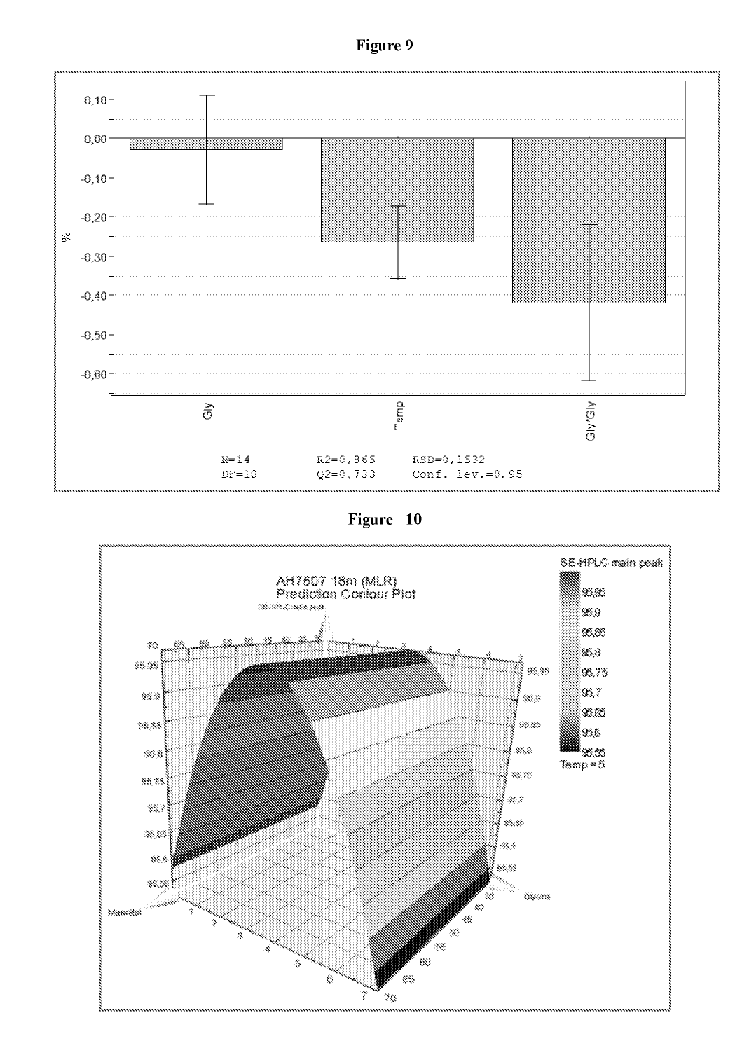

For the MLR model applied to data from samples stored at +5° C. and +25° C., FIG. 9 shows that the effect of the factors on the SE-HPLC main peak (rhBSSL monomers) showed that the storage temperature had, as expected, a negative effect (i.e., that an increase in storage temperature is associated with a decrease in rhBSSL monomers), but also that the square of the glycine factor contributed negatively, indicating a curvature in the model and suggesting the existence of an optimum glycine concentration. Glycine as a linear factor had no significant effect in itself on the SE-HPLC main peak response. The model was described by a R² of 0.865 and a Q² of 0.733, indicating both high proportion of variance accounted for by the factors, and good predictability.

The MLR model applied to data from samples stored at +5° C. was used to design another favorable formulation in regards to the amount of glycine with respect to a maximum in SE-HPLC main peak (rhBSSL monomers) using a contour plot, which determined that glycine may vary between 2.1 and 4.9 mg/vial and still result in a SE-HPLC main peak of ≧95.9% after 18 months at +5° C. The contour plot is presented in FIG. 10 where the glycine optimum can be seen. A similar determination was done for the results from storage at +25° C., producing approximately the same glycine optimum but slightly affected negatively the SE-HPLC main peak percentage at this storage temperate.

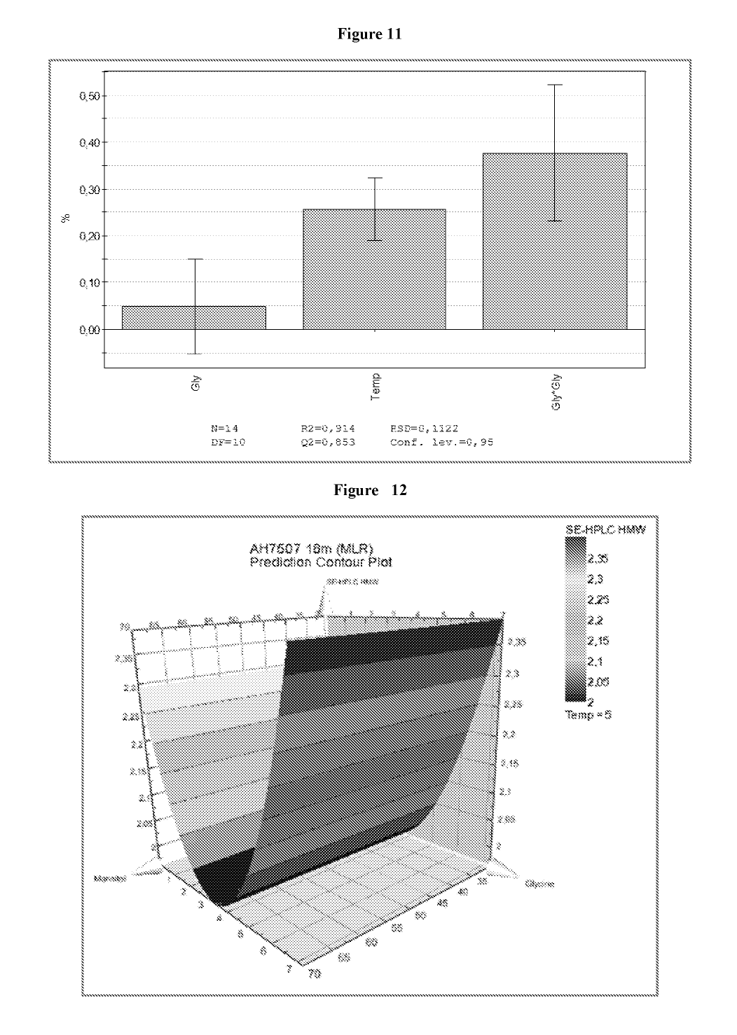

For the MLR model applied to data from samples stored at +5° C. and +25° C. with SE-HPLC HMW (amount of rhBSSL aggregates) as the response variable, the effect of the factors were close to the complete opposite of that observed for the SE-HPLC main peak (rhBSSL monomers). This is related to the fact that the SE-HPLC HMW and main peak are responses from the same analytical method and to a certain extent connected: an increase in rhBSSL aggregates will result in some reduction in rhBSSL monomers. Accordingly, in this model with SE-HPLC HMW (amount of rhBSSL aggregates) as the response variable, the storage temperature and the square of the glycine amount was seen to have a significant effect on the SE-HPLC HMW (rhBSSL aggregates) as seen in FIG. 11, with more aggregation forming at storage at higher temperatures and a curvature in the model with respects to glycine amount. This model describing rhBSSL aggregate formation after storage at +5° C. or +25° C. for 18 months accounted for a large portion of total variance (R² equal to 0.914) and a high predictability (Q² of 0.853).

The MLR model applied to data from samples stored at +5° C. was used to design a further favourable formulation in regards to the amount of glycine with respect to a minimum in SE-HPLC HMW peaks (rhBSSL aggregates) using a contour plot, which determined that glycine may vary between 2.3 and 4.5 mg/vial and still result in less than 2.0% rhBSSL aggregates (SE-HPLC HMW) after 18 months at +5° C. The contour plot is presented in FIG. 12 where the glycine optimum can be seen. A similar determination was done for storage at +25° C. which resulted in approximately the same glycine optimum but slight modifications in SE-HPLC HMW peak percentage at this storage temperate.

Lyophilization: A number of vials sufficient for each all storage conditions and sampling times were prepared for each formulation N1 to N7 by lyophilization of an appropriate liquid formulation comprising rhBSSL and the respective excipients in appropriate amounts and concentrations. The vials of the different liquid formulations were lyophilized, and unit-dose forms (vials) prepared of each lyophilized formulation N1 to N7, as generally described as follows: a liquid pre-formulation prepared as below is aliquoted into clear white 6 mL (6R) glass vials of ISO standard (Mglas), each containing 1.25 mL of liquid formulation, and batches of aliquoted vials are placed into a lyophilizer (LyoStar II, FTS systems). The samples are cooled to −50° C. at a rate of approximately 0.8° C./hour and let to equilibrate at −50° C. for 5 h, and primary drying is conducted by applying a vacuum of 0.3 mbar which is maintained for 62 hours with a shelf temperature of 0° C. During this time the temperature of a sample approaches the temperature of the shelf, indicating that sublimation of ice crystals is complete. Secondary drying is initiated by lowering the chamber pressure to 0.02 mbar and raising the temperature of the shelves to +10° C. at a rate of about 0.3° C./hour. Secondary drying is continued for about 20 hours until the product has a moisture content of between about 1% and 0.2%, whereupon the vials are sealed under vacuum with rubber stoppers (West Pharmaceutical Services).

Liquid formulation suitable for lyophilization: A batch of each liquid formulation used to prepare each of the lyophilized formulations N1 to N7 was analogously prepared and aliquoted into an appropriate number of vials prior to lyophilization, as generally described in the appropriate section of EXAMPLE 4, except that the final composition of the various liquid formulations prior to lyophilization was as described in Table 2 and that each vial was filled with 1.25 mL. The pH of each liquid formulation was typically found to be between 6.6 and 7.2.

TABLE 2
Composition of liquid formulation of rhBSSL suitable for forming
lyophilized formulations N1 to N7.

| | rhBSSL | | Sodium | Sodium | | |
|---|---|---|---|---|---|---|
| Sam- | enzyme | | Chloride | Phosphate* | Mannitol | Glycine |
| ple | activity | | mg/mL | mg/mL | mg/mL | mg/mL |
| no. | mg/mL | U/mL | (mM) | (mM) | (mM) | (mM) |
| N1 | 12.0 | 6948 | 1.46 (25) | 1.6 (12) | 24 (132) | 0.00 (0) |
| N2 | 12.0 | 6948 | 1.46 (25) | 1.6 (12) | 40 (220) | 0.00 (0) |
| N3 | 12.0 | 6948 | 1.46 (25) | 1.6 (12) | 56 (307) | 0.00 (0) |
| N4 | 12.0 | 6948 | 1.46 (25) | 1.6 (12) | 24 (132) | 2.45 (33) |
| N5 | 12.0 | 6948 | 1.46 (25) | 1.6 (12) | 56 (307) | 5.77 (77) |
| N6 | 12.0 | 6948 | 1.46 (25) | 1.6 (12) | 40 (220) | 2.06 (27) |
| N7 | 12.0 | 6948 | 1.46 (25) | 1.6 (12) | 40 (220) | 2.06 (27) |

*Sodium phosphate molar concentrations calculated from weighted-average molecular weights and mass of material of the two sodium phosphate components.

Storage conditions: The vials of each lyophilized formulation were randomized between storage at the 3 storage temperatures +5° C., +25° C. and +40° C., with the number of vials used for temperate sufficient for the sampling to be conducted over time. For each storage condition the vials were stored in a light-proof outer container or cabinet which was only opened when a vial was to be removed for analysis at a given time-point. The desired temperature was maintained within the following ranges: for +5° C. between +2° C. and +8° C.; for 25° C. between +23° C. and +27° C.; and for +0° C. between +38° C. and +42° C.

Analysis (i) PXRD: A sample of each lyophilized formulation N1 to N7 was subjected to powder X-ray diffraction (PXRD) analysis to investigate the crystalline state of the various components. Briefly, Briefly an X-ray powder diffraction analysis was carried out on a θ/2θ diffractometer (parafocusing Bragg-Brentano geometry) with sample spinning capability (X'pert PRO, Philips Analytical, Netherlands) using Cu Kα1 radiation (1.5406 Å). The samples were investigated from 5° to 50° 2θ at a step size of 0.013°. The diffractometer alignment was tested against NIST standard reference materials. Sample preparation: the lyophilized material was placed in a steel sample holder containing a zero-background silicon wafer and then covered with a Kaptone foil. Diffraction data were evaluated (indexing and Rietveld refinements) with standard crystallographic software including FULLPROF (Rodriguez-Carvajal, 2001Commission on Powder Diffraction (IUCr) Newsletter 26; 12-19), DICVOL06, TREOR90, X'Pert HighScore Plus and MDI JADE 8.

Analysis (ii) SE-HPLC: Upon removal of a vial from storage, the lyophilized formulation it contained was promptly reconstituted in 1.0 mL deionized water by agitation for about 5 min and subjected to size-exchange high-performance liquid chromatography to detect any changes in the amount of rhBSSL monomers (main peak) or rhBSSL aggregates (high molecular weight peaks). Briefly, size exclusion chromatography was carried out on a TSK-gel Super SW3000 30×4.6 mm (Tosoh Bioscience). The column was equilibrated and ran in 10 mM sodium phosphate, 0.4 M sodium chloride, pH 7, at a flow rate of 0.15 mL/min using a Agilent 1100 series HPLC equipped with a diode array detector. The sample was compared to a reference standard of pure rhBSSL The sample load was 1 μg and protein was detected by monitoring the UV absorption at 214 nm. The data was evaluated with the software Chemstation Plus and Chemstore (Agilent technologies).

### Example 2

### Experiments AH7513 and AH7517

Experimental set-up: The effect of glycine as an amorphous stabilizing agent on the properties of a lyophilized formulation of rhBSSL was further studied in two further experiments AH7513 and AH7517. Excipients in various combinations and amounts were used to produce a further 4 lyophilized formulations having the compositions presented in Table 3. The samples were stored for various times at +5° C., +25° C. and +40° C. At regular periods during storage, samples were taken from the various lyophilized formulations, and studied using size-exclusion high-performance liquid chromatography (SE-HPLC), powder X-ray diffraction (PXRD) and other techniques.

TABLE 3
Amount of rhBSSL and excipients in lyophilized powder of the lyophilized
formulations of AH7513 and AH7517.

| | | rhBSSL enzyme | | Sodium | Sodium | | |
|---|---|---|---|---|---|---|---|
| Experiment | Sample | activity | | Chloride | Phosphate* | Mannitol | Glycine |
| no. | no. | mg/vial | U/vial | mg/vial | mg/vial | mg/vial | mg/vial |
| AH7513 | F1 | 15.6 | 9032 | 1.30 | 1.7 | 45.0 | 5 |
| | F2 | 15.6 | 9032 | 1.30 | 1.7 | 45.0 | 0 |
| AH7517 | G2 | 15.6 | 9032 | 1.30 | 1.7 | 45.0 | 4.0 |
| | G3 | 15.6 | 9032 | 1.30 | 1.7 | 45.0 | 4.5 |

*Amount of sodium phosphate calculated from weighted-average molecular weights and mass of material of the two sodium phosphate components.

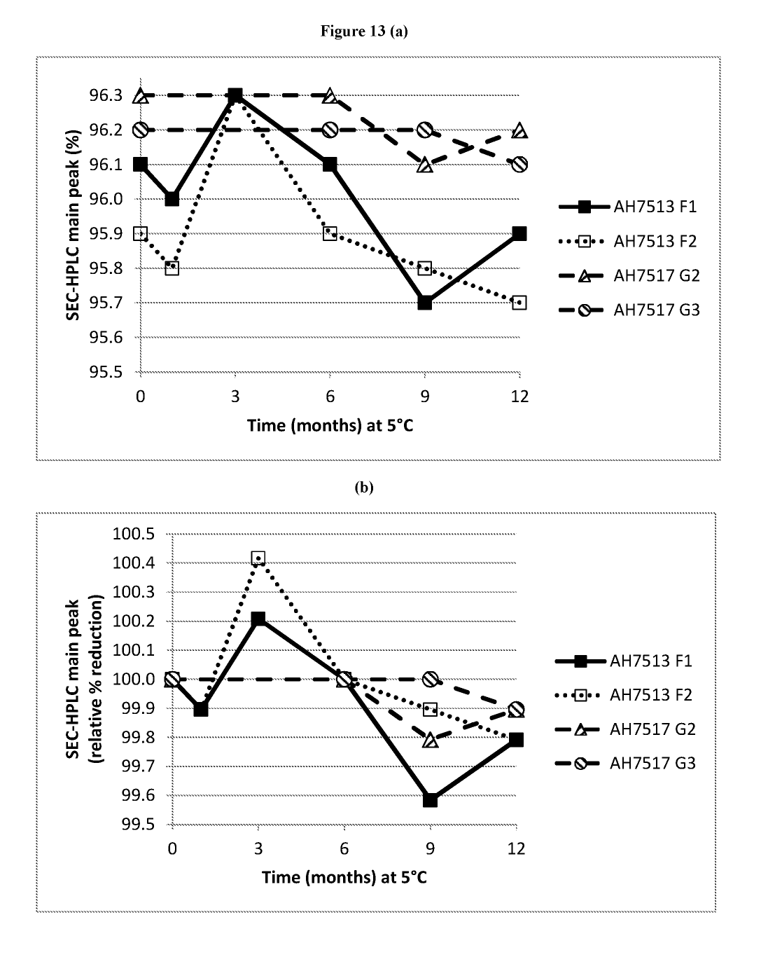

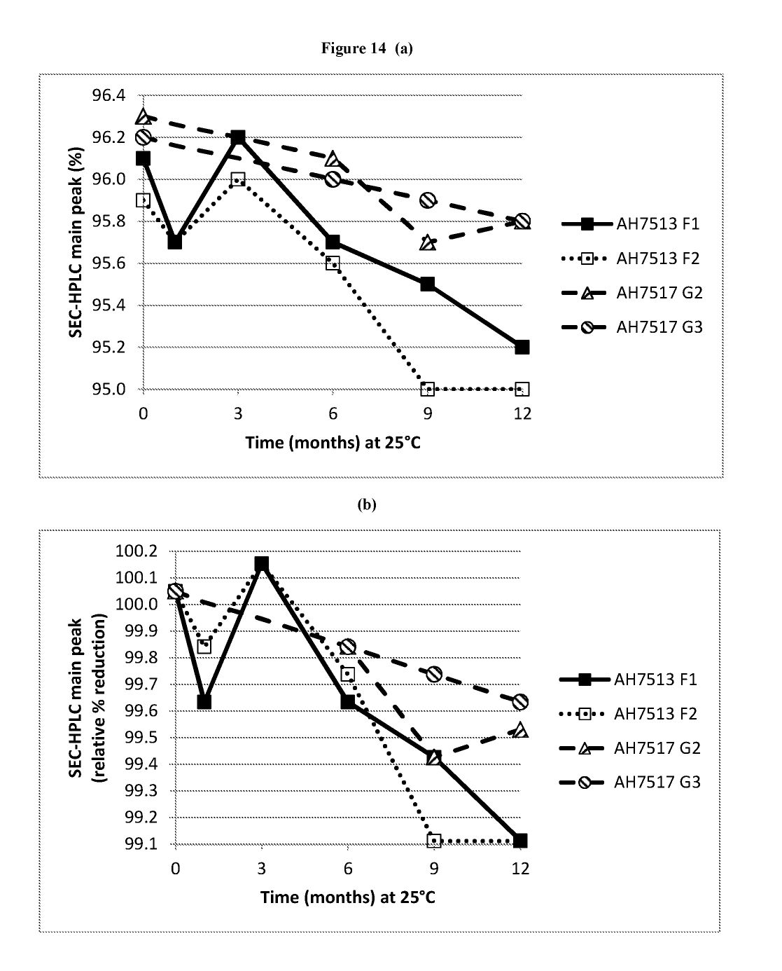

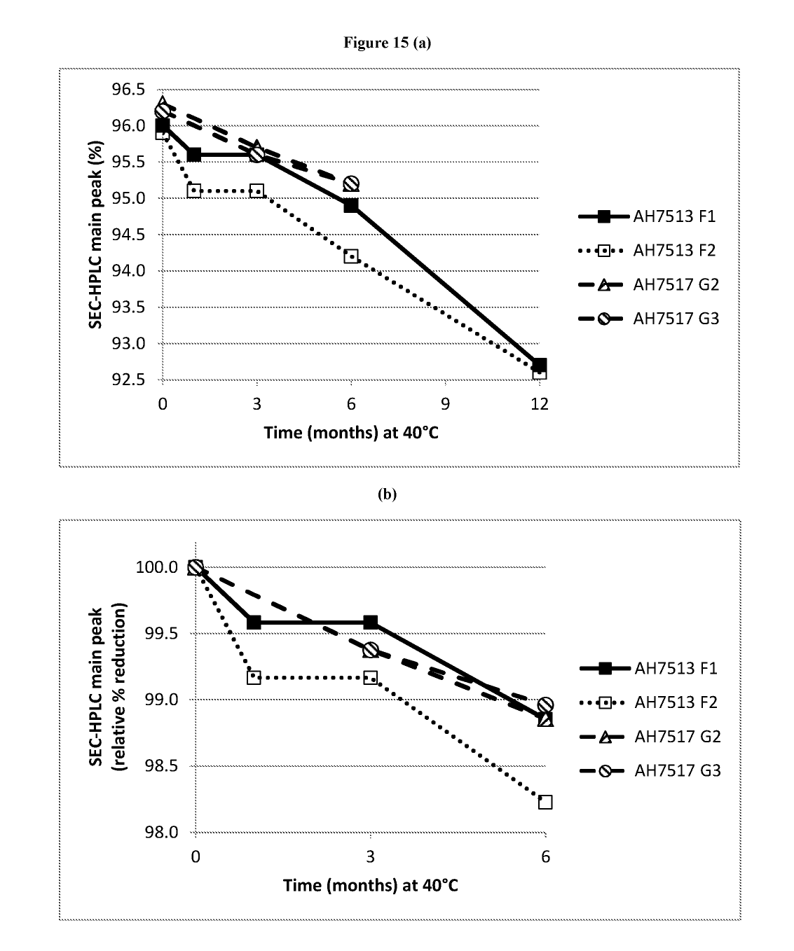

Results (i) monomerization and aggregation of rhBSSL studied by SE-HPC: During long term storage, rhBSSL in monomeric form was detected as main peak and the formation of rhBSSL aggregates detected as higher molecular-weight peaks were studied using SE-HPLC as described in EXAMPLE 1. FIG. 13 shows the amount of rhBSSL monomer (quantified by the main peak of SE-HPLC) present in the various lyophilized formulations after storage at +5° C. for various periods of time, FIG. 14 shows the same after storage at +25° C., and FIG. 15 shows the same after storage at +40° C.

In all figures FIG. 13 to FIG. 18, the general classes of concentration of glycine that was present in each liquid formulation prior to lyophilization is also indicated by the shading of the plotted symbols, with solid symbols representing a “High” glycine concentration of 56 mM, the open symbols representing a “Low” glycine concentration of 0 mM and the hatched symbols representing “Medium” glycine concentrations of 44 mM (for G2) and 50 mM (for G3). This coding aids the interpretation of these graphs with respect to the greater stability of the formulations that were obtained from the “Medium” glycine concentrations.

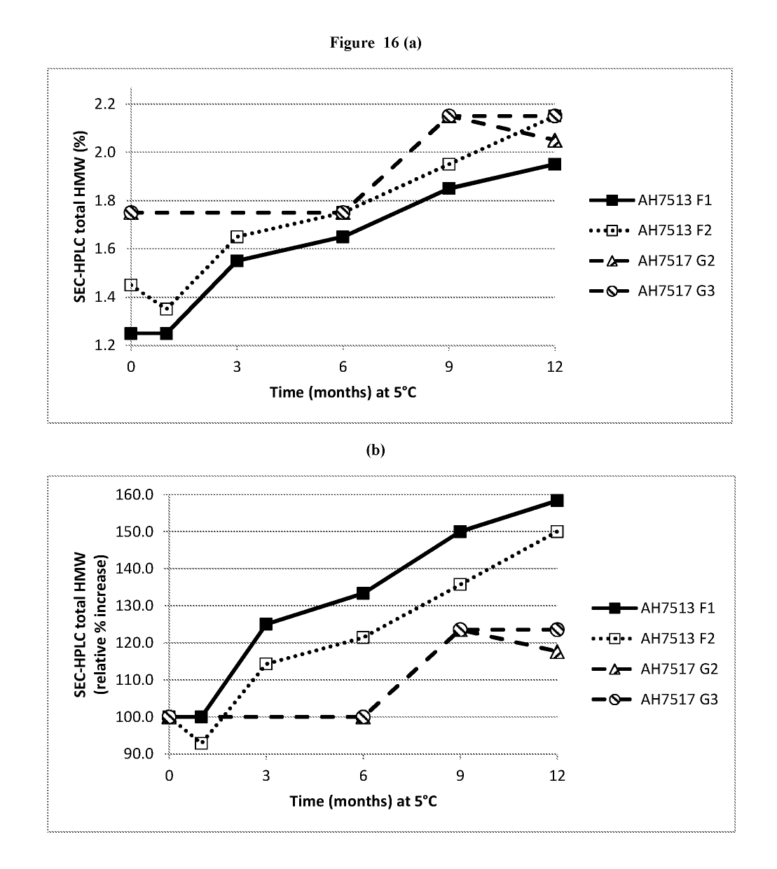

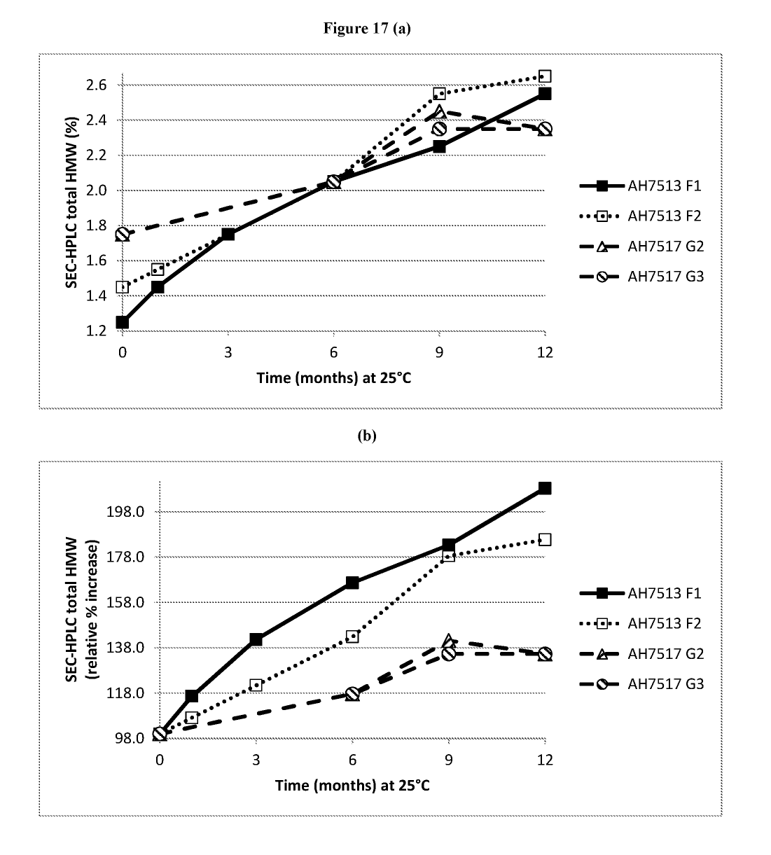

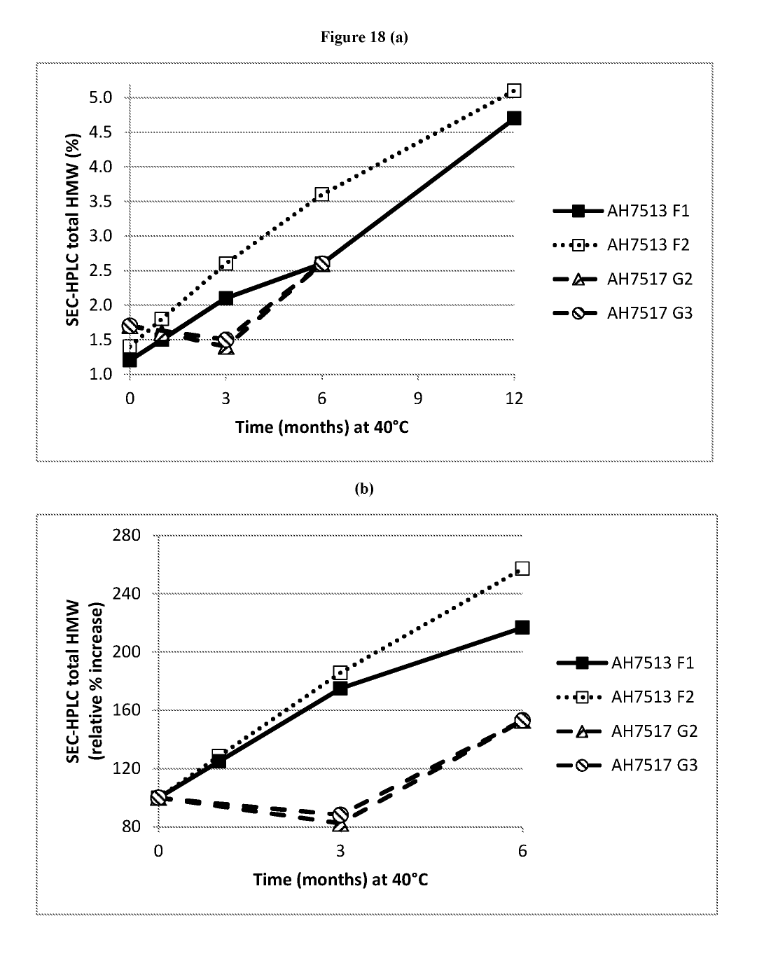

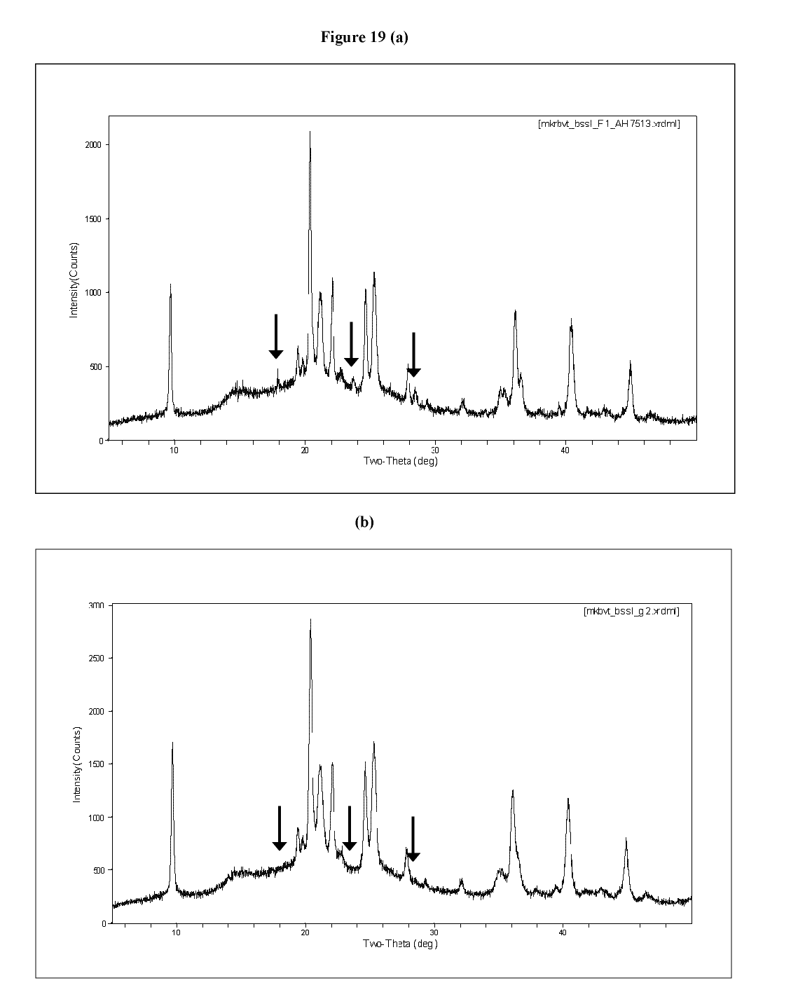

FIG. 16, FIG. 17 and FIG. 18 show reduced accumulation of rhBSSL aggregates in formulations G2 and G3. This effect is more marked at the higher storage temperatures.

Results (ii) crystallization of components of the formulation studied by PXRD: Powder X-ray diffraction (PXRD) was used to determine the crystalline form of the components of each formulation of experiments AH7513 and AH7517 at various time-points during the experiment, from time zero up to the final storage sampling as gee rally described in EXAMPLE 1.

Surprisingly, and as can be seen from FIG. 19, the glycine present in formulation F1 appeared to be present in crystalline form, while in formulations G2 or G3 (data not shown) no crystalline form of glycine could be detected by PXRD, and the glycine in such formulations appear to be in amorphous form. The presence of amorphous glycine in the formulations G2 and G3 correlates with, and confirm the advantageous properties first detected in EXAMPLE 1, reduced loss of rhBSSL monomers (FIG. 13 to FIG. 15) and reduced accumulation of rhBSSL (insoluble) aggregates (FIG. 16 to FIG. 18).

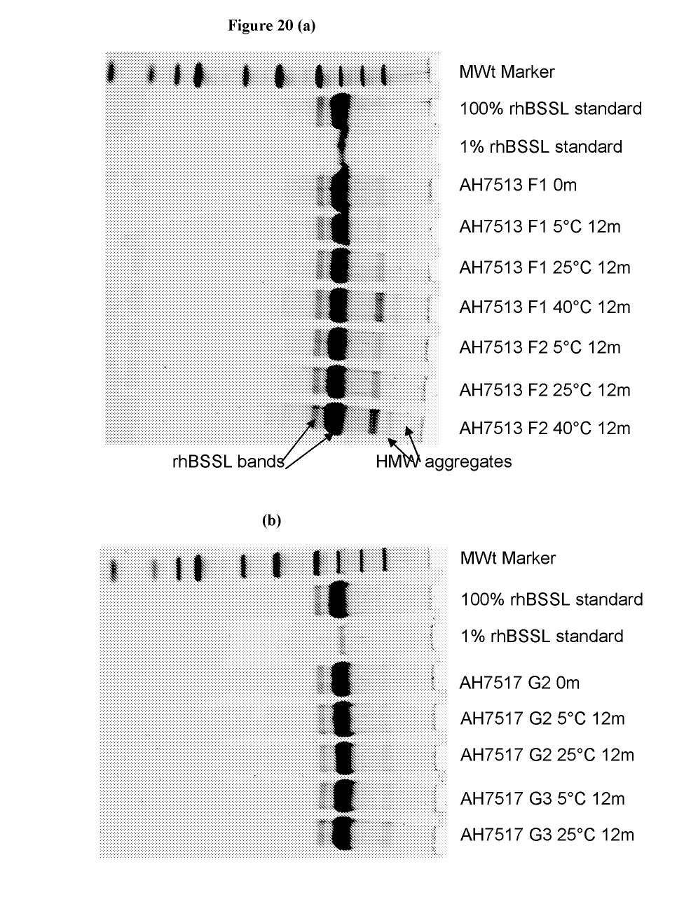

Results (iii) SDS-polyacrylamide gel electrophoresis: SDS-polyacrylamide gel electrophoresis (SDS-PAGE) is used to visually reveal rhBSSL aggregation. As shown in FIG. 20, the amount of HMW aggregates in formulations G2 and G3 after 12 months at +25° C. appears to be similar to that revealed in formulation F1 at time zero or F1 or F2 after storage at +5° C. for 12 months.

Lyophilization: A number of vials sufficient for each all storage conditions and sampling times were prepared for each formulation F1, F2, G1 and G2 by lyophilization of an appropriate liquid formulation comprising rhBSSL and the respective excipients in appropriate amounts and concentrations. The vials of the different liquid formulations were lyophilized, and unit-dose forms (vials) prepared of each lyophilized formulation F1, F2, G1 and G2, as generally described in the appropriate section of EXAMPLE 1 but using a fill-volume per vial of 1.20 mL.

Liquid formulation suitable for lyophilization: A batch of each liquid formulation used to prepare each of the lyophilized formulations F1, F2, G1 and G2 was analogously prepared and aliquoted into an appropriate number of vials prior to lyophilization, as generally described in the appropriate section of EXAMPLE 4, except that the final composition of the various liquid formulations prior to lyophilization was as described in Table 4 and that each vial was filled with 1.20 mL. The pH of each liquid formulation was typically found to be between 6.6 and 7.2

TABLE 4
Composition of liquid formulation of rhBSSL suitable for forming lyophilized
formulations F1, F2, G1 and G2.

| | | rhBSSL | | | | | |
|---|---|---|---|---|---|---|---|
| | | enzyme | | Sodium | Sodium | | |
| Experiment | | activity | | Chloride | Phosphate* | Mannitol | Glycine |
| no. | Sample no. | mg/mL | U/mL | mg/mL (mM) | mg/mL (mM) | mg/mL (mM) | mg/mL (mM) |
| AH7513 | F1 | 13.0 | 7527 | 1.08 (18.5) | 1.6 (12) | 37.5 (206) | 4.2 (56) |
| | F2 | 13.0 | 7527 | 1.08 (18.5) | 1.6 (12) | 37.5 (206) | 0 (0) |
| AH7517 | G2 | 13.0 | 7527 | 1.08 (18.5) | 1.6 (12) | 37.5 (206) | 3.3 (44) |
| | G3 | 13.0 | 7527 | 1.08 (18.5) | 1.6 (12) | 37.5 (206) | 3.75 (50) |

*Sodium phosphate molar concentrations calculated using weighted-average molecular weights and mass of material of the two sodium phosphate components.

Analysis: samples of the lyophilized formulations F1, F2, G1 and G2 were analyzed using liquid chromatography (SE-HPLC), powder X-ray diffraction (PXRD) as generally described in EXAMPLE 1. SDS-PAGE was conducted using standard procedures within a 4-12% gradient PA gel. Sample was dissolved in lithium dodecyl sulphate buffer (LDS) at a LDS concentration of 1% or approx. 40 μg/μg protein (0.25 μg protein/mL in the sample).

### Example 3

### Comparison to a Liquid Formulation of rhBSSL

By way of further evidence of the superiority of the lyophilized formulations of the present invention, the liquid formulation of rhBSSL (the drug-substance (DS) produced as described in EXAMPLE 4 below) was subjected to analogous stability studies by storage for up to 3 months at +25° C. After 3 months storage at +25° C., the SE-HPLC of the DS showed 93.3% main peak (rhBSSL monomers) and 3.6% total integrated HMW peaks (representing rhBSSL aggregates). This is surprisingly less stable than any of the lyophilized formulations of the present invention. For example, even formulation N1 of experiment AH7505 (containing no glycine but a “Low” amount of mannitol) did not lose as much % main peak or generate as much % HMW peaks, even after 18 months storage at the same temperature (see FIG. 2 and FIG. 5 respectively). Furthermore, the optimized lyophilized formulations G2 and G3, despite storage at +40° C. for 6 months, still retained around 95% main peak and not more than about 2.6% total HMW peaks.

### Example 4

### An Optimized Lyophilized Formulation for rhBSSL and a Unit Dose Thereof

Based on the results of experiments such as above, one optimized lyophilized formulation of rhBSSL is prepared as described below, with all steps conducted under GMP conditions.

Drug substance production: The drug substance, human bile salt-stimulated lipase, having a predicted amino acid sequence as shown in SEQ ID NO: 2, is produced, for example, by expression from recombinant Chinese hamster ovary (CHO) cells containing a nucleic acid expression system comprising the nucleotide sequence encoding human BSSL according to standard procedures.

By way of brief description for such a process, the 2.3Kb cDNA sequence encoding full-length hBSSL including the leader sequence (as described by Nilsson et al, 1990; Eur J Biochem, 192: 543-550) is obtained from pS146 (Hansson et al, 1993; J Biol Chem, 268: 26692-26698) and cloned into the expression vector pAD-CMV 1 (Boehringer Ingelheim)—a pBR-based plasmid that includes CMV promoter/SV40 polyA signal for gene expression and the dhfr gene for selection/amplification—to form pAD-CMV-BSSL. pAD-CMV-BSSL is then used for transfection of DHFR-negative CHOss cells (Boehringer Ingelheim)—together with co-transfection of plasmid pBR3127 SV/Neo pA coding for neomycin resistance to select for geneticin (G418) resistance—to generate DHFR-positive BSSL producing CHO cells. The resulting CHO cells are cultured under conditions and scale to express larger quantities of rhBSSL. For example, cells from the master cell bank (MCB) are thawed, expanded in shaker flasks using Ex-Cell 302 medium without glutamine and glucose (SAFC) later supplemented with glutamine and glucose, followed by growth in 15 and 100 L bioreactors, before inoculating the 700 L production bioreactor where BSSL is constitutively expressed and produced in a fed-batch process. The culture is harvested as a single batch and the mature rhBSSL polypeptide (i.e., without the leader sequence) is purified from cells, cell debris and other contaminates via a number of downstream steps, including an anion exchange chromatography step. Contaminating viruses may be inactivated by low pH treatment and a dry heat treatment step. The rhBSSL Drug Substance (DS) bulk is diafiltered and concentrated to approximately 20-25 mg/mL.

The specific activity of the bulk DS is determined using 4-nitrophenyl ester butyric acid (PNPB) as a substrate for BSSL, and detection of the release of 4-nitrophenol. Briefly, a dilution series of rhBSSL (for example, from 20 to 160 ng activity/mL) is prepared in PBS with 0.1% BSA. 200 μl of these rhBSSL solutions is added to 25 μl of an activation solution containing 20 mM sodium cholate (as bile salt activator) in PBS with 0.1% BSA. These solutions are preincubated in a spectrophotometer at +27° C. for 5 minutes. Just before measuring, 25 μl of a well-mixed substrate solution containing 5 mM PNPB in PBS-Tween is added. The formation of 4-nitrophenol can be detected by its absorbance at 400 nm and the increase in absorbance is measured during 90 seconds. The active amount of BSSL is determined using a standard curve of an rhBSSL reference standard

Liquid formulation suitable for lyophilization: The concentration of the solution of bulk rhBSSL DS is adjusted to 10130 U/mL with 10 mM Sodium phosphate, 25 mM Sodium chloride, pH 7 in an around 300 L vessel with stirring, equipped with magnetic stirrer, and the excipients listed in

Table 5 are added to the final concentration shown to form a liquid formulation of rhBSSL suitable for lyophilization. The pH of such a formulation is typically between 6.6 and 7.2.

TABLE 5
Composition of a liquid formulation
of rhBSSL suitable for lyophilization

| | Molar | | |
|---|---|---|---|
| | concen- | Mass | |
| | tration | concen- | Units for mass |
| Component | (mM) | tration | concentration |
| rhBSSL | N/A | 7504 (13.0) | U/mL (mg/mL) |
| Disodium hydrogen | 5.5 | 0.98 (0.09%) | mg/mL (w/v %) |
| phosphate dehydrate* | | | |
| Sodium dihydrogen | 4.5 | 0.62 (0.07%) | mg/mL (w/v %) |
| phosphate monohydrate* | | | |
| Sodium chloride | 19 | 1.08 (0.11%) | mg/mL (w/v %) |
| Mannitol | 205 | 37.4 (3.74%) | mg/mL (w/v %) |
| Glycine | 44 | 3.32 (0.33) | mg/mL (w/v %) |

*Sodium phosphate molar concentrations calculated using molecular weights and mass of material of the respective sodium phosphate component.

Lyophilization and preparation of unit doses: The liquid formulation prepared above is aliquoted into clear white 6 mL (6R) glass vials of ISO standard (Sofferia Bertolini), each containing 1.26 mL of liquid formulation, and batches of aliquoted vials are placed into a lyophilizer (Lyomax 33, BOC Edwards). The samples are cooled to −50° C. at a rate of approximately 0.8° C./hour and let to equilibrate at −50° C. for 5 h, and primary drying is conducted by applying a vacuum of 0.2 mbar which is maintained for 13 hours with a shelf temperature of 0° C. During this time the temperature of a sample approaches the temperature of the shelf, indicating that sublimation of ice crystals is complete. Secondary drying is initiated by lowering the chamber pressure to 0.02 mbar and raising the temperature of the shelves to +25° C. at a rate of about 1° C./hour. Secondary drying is continued for about 10 hours until the product has a moisture content of between about 0.8% and 0.2%, whereupon the vials are sealed under vacuum with rubber stoppers (West Pharmaceutical Services).

Each vial produced as describe above is a unit dose form of a lyophilized formulation comprising rhBSSL with the components and in the amounts as listed in

Table 6. Powder X-ray diffraction (as described in EXAMPLE 1) of this formulation shows no evidence of glycine crystals (see FIG. 15), and also no evidence of sodium phosphate or sodium chloride crystals.

TABLE 6
Composition per vial (unit dose form) of an optimized
lyophilized formulation comprising rhBSSL

| Component | Amount (/vial) | |
|---|---|---|
| rhBSSL | 9478 U (16.4 mg) | |
| Disodium hydrogen phosphate dihydrate | 1.1 | mg* |
| Sodium dihydrogen phosphate monohydrate | 0.9 | mg* |
| Sodium chloride | 1.4 | mg |
| Mannitol | 47 | mg |
| Glycine | 4.2 | mg |

*Amounts given as mass of the respective sodium phosphate component.

By minor variation of the concentrations of the excipients in the pre-formulation and the fill-volume, now within the experience of the person of ordinary skill following disclosure of the present invention, other optimized lyophilized formulations comprising rhBSSL are formed. For example, the formulation comprising rhBSSL with the components and in the amounts as listed in Table 7.

TABLE 7
Composition per vial (unit dose form) of an optimized
lyophilized formulation comprising rhBSSL

| Component | Amount (/vial) | |
|---|---|---|
| rhBSSL | 10027 | U |
| Disodium hydrogen phosphate dihydrate | 1.0 | mg* |
| Sodium dihydrogen phosphate monohydrate | 0.7 | mg* |
| Sodium chloride | 1.4 | mg |
| Mannitol | 50 | mg |
| Glycine | 4.4 | mg |

*Amounts given as mass of the respective sodium phosphate component.

The absolute composition per vial (unit dose form) may depend on the dose-rate to be administered, and particularly if administered to pre-term infants, lower absolute amounts of rhBSSL per vial may be desired, while keeping about the same relative composition of the excipients. This may be achieved, for example, by using a small-fill volume (such as about half of the volumes given above) of the same liquid pre-formulation, and in certain instances (such as to differentiate dose-amounts) using smaller or different colored vials. Accordingly, the composition of an optimized lyophilized formulations comprising rhBSSL (such as the one shown above) may be represented relative to e.g. the amount of rhBSSL present in the formulation, such as the mg amount of each excipient per 10,000 U or rhBSSL.

TABLE 8
Relative composition per vial (unit dose form)
of an optimized lyophilized formulation comprising
rhBSSL in mg/10,000 U of rhBSSL

| | Relative amount |
|---|---|
| Component | (mg/10,000 U rhBSSL) |
| Disodium hydrogen phosphate dihydrate | 1.0 |
| Sodium dihydrogen phosphate monohydrate | 0.7 |
| Sodium chloride | 1.4 |
| Mannitol | 50 |
| Glycine | 4.4 |

* Relative amounts based on the mass of the respective sodium phosphate component.

Reconstitution of the lyophilized formulation: The unit dose described above is reconstituted in a liquid infant feed, for example by addition and shaking to dissolve such formulation in 100 mL of pasteurized breast milk of infant formula. Such a liquid infant feed containing rhBSSL can be conveniently used to administer an effective amount of rhBSSL to an infant in need of treatment therewith, such as a preterm infant Such administration may occur orally by feeding with a bottle, or via the GI tract by using nasal feeding. The pH of such a liquid infant feed comprising rhBSSL reconstituted from a lyophilized formulation of the invention is typically found to be between 6.4 and 7.4.

As an alternative to a liquid infant feed, multiple the unit dose forms (or a unit dose comprising a larger amount of each component) is analogously reconstituted in fruit juice or water. rhBSSL reconstituted in such a manner can be conveniently used to orally administer an effective amount of rhBSSL to children or adults, such as those suffering from pancreatic insufficiency, in particular that caused by cystic fibrosis.

1. A formulation suitable for lyophilization comprising:
(i) recombinant human bile salt-stimulated lipase (rhBSSL);
(ii) a crystalline bulking agent; and
(iii) an amorphous stabilizer that is a different chemical entity to said crystalline bulking agent and is present at a relative amount of between about 0.1 mg and about 0.5 mg per mg of said rhBSSL.

2. The formulation of claim 1, wherein said amorphous stabilizer is selected from the group consisting of: L-arginine; L-histidine; L-proline; L-alanine; and glycine.

3. The formulation of claim 2, wherein said amorphous stabilizer is glycine.

4. The formulation of claim 1, wherein said amorphous stabilizer is present at a concentration of between 10 mM and 100 mM.

5. The formulation of claim 4, wherein said amorphous stabilizer is present at a concentration of between 35 mM and 50 mM.

6. The formulation of claim 1, wherein said crystalline bulking agent is mannitol.

7. The formulation of claim 1, wherein said crystalline bulking agent is present at a concentration of between 100 mM and 400 mM.

8. The formulation of claim 7, wherein said crystalline bulking agent is present at a concentration of between 180 mM and 210 mM.

9. The formulation of claim 1, wherein said rhBSSL is present at a concentration of between 1 mg/mL and 35 mg/mL.

10. The formulation of claim 9, wherein said rhBSSL is present at a concentration of between 10 mg/mL and 15 mg/mL.

11. The formulation of claim 10, comprising:
(i) rhBSSL present at a concentration of between 10 mg/mL and 15 mg/mL;
(ii) mannitol present at a concentration of between 180 mM and 210 mM; and
(iii) glycine present at a concentration of between 35 mM and 50 mM.

12. The formulation of claim 1, having a pH value of between 6.3 and 7.5.

13. The formulation of claim 1, further comprising sodium phosphate, present at a phosphate concentration of between 2 mM and 20 mM.

14. The formulation of claim 1, further comprising sodium chloride, present at a chloride concentration of between 5 mM and 50 mM.

15. A lyophilized formulation obtainable by lyophilization of a formulation of claim 1.

16. A reconstituted formulation of rhBSSL, comprising:
(i) said rhBSSL present in an absolute amount of between 10 mg and 20 mg;
(ii) mannitol present in an absolute amount of between 27 mg and 62 mg;
(iii) glycine present in an absolute amount of between 2 mg and 6 mg;
and wherein said formulation is reconstituted in a liquid infant feed; and said reconstituted formulation has a pH of between 6.4 and 7.4.

17. Use of glycine to stabilize rhBSSL, present in a lyophilized formulation further comprising a crystalline bulking agent that is not glycine, wherein said glycine is present in said lyophilized formulation in non-crystalline form; and/or said glycine is included in said lyophilized formulation at a relative amount of between 0.2 mg and 0.3 mg per mg of said rhBSSL.


SEQUENCE LISTING (2 sequences)

SEQ ID NO: 1 - DNA, 2428 bp, Homo sapiens
  CDS (151)..(2319)
accttctgtatcagttaagtgtcaagatggaaggaacagcagtctcaagataatgcaaagagtttattcatccagaggctgatgctcaccatggggcgcctgcaactggttgtgttgggcctcacctgctgctgggcagtggcgagtgccgcgaagctgggcgccgtgtacacagaaggtgggttcgtggaaggcgtcaataagaagctcggcctcctgggtgactctgtggacatcttcaagggcatccccttcgcagctcccaccaaggccctggaaaatcctcagccacatcctggctggcaagggaccctgaaggccaagaacttcaagaagagatgcctgcaggccaccatcacccaggacagcacctacggggatgaagactgcctgtacctcaacatttgggtgccccagggcaggaagcaagtctcccgggacctgcccgttatgatctggatctatggaggcgccttcctcatggggtccggccatggggccaacttcctcaacaactacctgtatgacggcgaggagatcgccacacgcggaaacgtcatcgtggtcaccttcaactaccgtgtcggcccccttgggttcctcagcactggggacgccaatctgccaggtaactatggccttcgggatcagcacatggccattgcttgggtgaagaggaatatcgcggccttcgggggggaccccaacaacatcacgctcttcggggagtctgctggaggtgccagcgtctctctgcagaccctctccccctacaacaagggcctcatccggcgagccatcagccagagcggcgtggccctgagtccctgggtcatccagaaaaacccactcttctgggccaaaaaggtggctgagaaggtgggttgccctgtgggtgatgccgccaggatggcccagtgtctgaaggttactgatccccgagccctgacgctggcctataaggtgccgctggcaggcctggagtaccccatgctgcactatgtgggcttcgtccctgtcattgatggagacttcatccccgctgacccgatcaacctgtacgccaacgccgccgacatcgactatatagcaggcaccaacaacatggacggccacatcttcgccagcatcgacatgcctgccatcaacaagggcaacaagaaagtcacggaggaggacttctacaagctggtcagtgagttcacaatcaccaaggggctcagaggcgccaagacgacctttgatgtctacaccgagtcctgggcccaggacccatcccaggagaataagaagaagactgtggtggactttgagaccgatgtcctcttcctggtgcccaccgagattgccctagcccagcacagagccaatgccaagagtgccaagacctacgcctacctgttttcccatccctctcggatgcccgtctaccccaaatgggtgggggccgaccatgcagatgacattcagtacgttttcgggaagcccttcgccacccccacgggctaccggccccaagacaggacagtctctaaggccatgatcgcctactggaccaactttgccaaaacaggggaccccaacatgggcgactcggctgtgcccacacactgggaaccctacactacggaaaacagcggctacctggagatcaccaagaagatgggcagcagctccatgaagcggagcctgagaaccaacttcctgcgctactggaccctcacctatctggcgctgcccacagtgaccgaccaggaggccacccctgtgccccccacaggggactccgaggccactcccgtgccccccacgggtgactccgagaccgcccccgtgccgcccacgggtgactccggggccccccccgtgccgcccacgggtgactccggggccccccccgtgccgcccacgggtgactccggggccccccccgtgccgcccacgggtgactccggggccccccccgtgccgcccacgggtgactccggggccccccccgtgccgcccacgggtgactccggggccccccccgtgccgcccacgggtgactccggcgccccccccgtgccgcccacgggtgacgccgggcccccccccgtgccgcccacgggtgactccggcgccccccccgtgccgcccacgggtgactccggggccccccccgtgacccccacgggtgactccgagaccgcccccgtgccgcccacgggtgactccggggccccccctgtgccccccacgggtgactctgaggctgcccctgtgccccccacagatgactccaaggaagctcagatgcctgcagtcattaggttttagcgtcccatgagccttggtatcaagaggccacaagagtgggaccccaggggctcccctcccatcttgagctcttcctgaataaagcctcatacccctaaaaaaaaaaaaa

SEQ ID NO: 2 - protein, 722 aa, Homo sapiens
AKLGAVYTEGGFVEGVNKKLGLLGDSVDIFKGIPFAAPTKALENPQPHPGWQGTLKAKNFKKRCLQATITQDSTYGDEDCLYLNIWVPQGRKQVSRDLPVMIWIYGGAFLMGSGHGANFLNNYLYDGEEIATRGNVIVVTFNYRVGPLGFLSTGDANLPGNYGLRDQHMAIAWVKRNIAAFGGDPNNITLFGESAGGASVSLQTLSPYNKGLIRRAISQSGVALSPWVIQKNPLFWAKKVAEKVGCPVGDAARMAQCLKVTDPRALTLAYKVPLAGLEYPMLHYVGFVPVIDGDFIPADPINLYANAADIDYIAGTNNMDGHIFASIDMPAINKGNKKVTEEDFYKLVSEFTITKGLRGAKTTFDVYTESWAQDPSQENKKKTVVDFETDVLFLVPTEIALAQHRANAKSAKTYAYLFSHPSRMPVYPKWVGADHADDIQYVFGKPFATPTGYRPQDRTVSKAMIAYWTNFAKTGDPNMGDSAVPTHWEPYTTENSGYLEITKKMGSSSMKRSLRTNFLRYWTLTYLALPTVTDQEATPVPPTGDSEATPVPPTGDSETAPVPPTGDSGAPPVPPTGDSGAPPVPPTGDSGAPPVPPTGDSGAPPVPPTGDSGAPPVPPTGDSGAPPVPPTGDSGAPPVPPTGDAGPPPVPPTGDSGAPPVPPTGDSGAPPVTPTGDSETAPVPPTGDSGAPPVPPTGDSEAAPVPPTDDSKEAQMPAVIRF